(19) United States
(12) Patent Application Publication
(10) Pub. No.: US 2024/0216392 A1
(43) Pub. Date: Jul. 4, 2024

(54) METHODS OF TREAMENT

(71) Applicant: Arena Pharmaceuticals, Inc., New York, NY (US)

(72) Inventors: Chadwick J. Orevillo, Lebanon, NJ (US); Randall Kaye, New York City, NY (US); Nuggehally Srinivas, Exton, PA (US)

(21) Appl. No.: 18/524,124

(22) Filed: Nov. 30, 2023

Related U.S. Application Data
(60) Provisional application No. 63/429,755, filed on Dec. 2, 2022.
(60) Provisional application No. 63/589,283, filed on Oct. 10, 2023.

(51) Int. Cl. A61K 31/5517; A61P 25/08

(52) U.S. Cl. CPC A61K 31/5517; A61P 25/08

(57) ABSTRACT

Provided herein are treatment methods including administering a 5-hydroxytryptamine (HT)_{2C} receptor agonist to a patient in need thereof. An exemplary method includes treating or preventing a 5-hydroxytryptamine (HT)_{2C} receptor-associated disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered twice daily.

### CROSS-REFERENCE TO RELATED APPLICATIONS

This application claims priority to U.S. Provisional Application No. 63/429,755, filed on Dec. 2, 2022, and U.S. Provisional Application No. 63/589,283, filed on Oct. 10, 2023, the disclosures of which are incorporated herein by reference in their entirety.

### TECHNICAL FIELD

The present application provides treatment and prevention methods including administering a 5-hydroxytryptamine (HT)_{2C} receptor agonist to a patient in need thereof.

### BACKGROUND

The developmental and epileptic encephalopathies (DEE) are a heterogeneous group of rare neurodevelopmental disorders. They are characterized by early-onset seizures that are often intractable and are associated with electroencephalographic abnormalities, and developmental delay or regression that can worsen over time. These disorders are generally diagnosed in childhood and adolescence; they vary in their etiologies, seizure types, electroencephalographic patterns, cognitive deficits, and prognosis. The International League Against Epilepsy recently expanded this definition to include disorders that may result in developmental delay before epilepsy onset and used the term of DEE to encompass this broader population.

5-HT2 receptor agonists have been shown to be efficacious treatments for a variety of motor seizures and seizure disorders. Specifically, low dose fenfluramine (Fintepla®), a mixed 5-HT_{2C}, 5-HT_{2B}, and 5-HT_{2A} receptor agonist, has recently been approved for the treatment of Dravet syndrome and Lennox-Gastaut Syndrome. However, Fintepla® received a Boxed Warning requiring cardiac monitoring because of the association between serotonergic drugs with 5-HT_{2B} receptor agonist activity and valvular heart disease.

The 5-HT_{2C} receptor agonist (R)—N-(2,2-difluoroethyl)-7-methyl-1,2,3,4,6,7-hexahydro-[1,4]diazepino[6,7,1-hi]indole-8-carboxamide (see U.S. Pat. No. 10,392,390) is in clinical trials for the treatment of DEE and related seizure disorders. US Pat. App. Pub. 2023/0293546 sets out TID dosing regimens (including up-titration and down-titration) at dosage amounts of 3, 6, 12, 18, and 24 mg and reports PK data including a half-life at steady state ranging from 4.81 to 6.50 hours.

There is a significant unmet need for safe and effective treatments for DEE and other seizure disorders. The compound and dosing methods described herein help satisfy this need and provide related advantages as well.

### SUMMARY

Provided is a method of treating or preventing a 5-hydroxytryptamine (HT)_{2C} receptor-associated disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered twice daily.

Also provided is a method of treating or preventing epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered twice daily.

Also provided is a method of reducing severity of an epileptic seizure in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered twice daily.

Also provided is a method of reducing the frequency of epileptic seizures in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered twice daily.

Also provided is a method of treating or preventing a seizure disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered twice daily.

Also provided is a method of treating or preventing developmental and epileptic encephalopathy (DEE) in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered twice daily.

Also provided is a method of treating or preventing a refractory epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof, is administered twice daily.

These and other aspects of the invention disclosed herein will be set forth in greater detail as the patent disclosure proceeds.

### BRIEF DESCRIPTION OF THE DRAWINGS

FIG. 1 is a graph showing PK (pharmacokinetic) profiles of Compound 1 in plasma and CSF (cerebrospinal fluid) following TID administration of 6 mg/dose of Compound 1.

FIG. 2 is a graph showing PK profiles of Compound 1 in plasma and CSF following TID administration of 12 mg/dose of Compound 1.

FIG. 3 is a set of plots of Compound 1 concentration in CSF and plasma following TID administration of 6 mg/dose (subjects 101-110) or 12 mg/dose (subjects 201-210) of Compound 1.

FIG. 4 is a plot comparing Cₘₐₓ of Compound 1 in CSF and plasma for TID administration of Compound 1.

FIG. 5 is a plot comparing AUCₜₐᵤ of Compound 1 in CSF and plasma for TID administration of Compound 1.

FIG. 6 is a table showing qEEG results following TID administration of 6 mg/dose of Compound 1.

FIG. 7 is a table showing qEEG results following TID administration of 12 mg/dose of Compound 1.

FIG. 8 is table showing PK characteristics for Compound 1 when administered with quinidine.

FIG. 9 is a set of plots showing mean concentration profiles of Compound 1 in plasma after BID and TID administration of Compound 1.

FIG. 10 is a set of plots showing mean concentration profiles of Compound 1 in CSF after BID and TID administration of Compound 1.

FIG. 11 is a plot comparing Cₘₐₓ of Compound 1 in CSF and plasma for BID administration of Compound 1.

FIG. 12 is a plot comparing AUCₜₐᵤ of Compound 1 in CSF and plasma for BID administration of Compound 1.

FIG. 13 is a set of plots comparing plasma average concentration and plasma maximum concentration of Compound 1 for 12 mg TID and 18 mg BID administration of Compound 1.

FIG. 14 is a set of plots comparing mean concentration of Compound 1 in plasma and CSF to Kᵢ for 5-HT_{2C} agonism.

FIG. 15 is a set of plots comparing CSF average concentration and CSF maximum concentration of Compound 1 for 12 mg TID and 18 mg BID administration of Compound 1.

FIG. 16 is a set of plots comparing percentage of time above Kᵢ in a dosing interval, for BID and TID dosing regimens.

FIG. 17 is a graph comparing CSF/P C_{trough} for 12 mg TID and 18 mg BID administration of Compound 1.

FIGS. 18A-18E are tables showing qEEG results for TID and BID administration of Compound 1.

FIGS. 19A-19E are tables showing qEEG results for TID and BID administration of Compound 1.

FIGS. 20A-20E are tables showing qEEG results for TID and BID administration of Compound 1.

FIGS. 21A-21E are tables showing qEEG results for TID and BID administration of Compound 1.

### DETAILED DESCRIPTION

As used in the present specification, the following words and phrases are generally intended to have the meanings as set forth below, except to the extent that the context in which they are used indicates otherwise.

### COMPOUND 1: As used herein, “Compound 1” means (R)—N-(2,2-difluoroethyl)-7-methyl-1,2,3,4,6,7-hexahydro-[1,4]diazepino[6,7,1-hi]indole-8-carboxamide

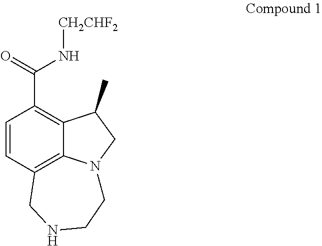

Compound 1, or a pharmaceutically acceptable salt thereof, is a potent and selective 5-hydroxytryptamine (HT)_{2C} receptor agonist and exhibits increased selectivity for the ligand binding site of 5-HT_{2C} receptors versus those of 5-HT_{2A} and 5-HT_{2B}. Compound 1 displays a binding affinity of 44 nM at the human 5-HT_{2C} receptor and shows no activity for 5-HT_{2A} and 5-HT_{2B}, in contrast, for example, to previously developed agonists such as Fintepla® (low dose fenfluramine).

Methods of use of Compound 1, or a pharmaceutically acceptable salt thereof, are disclosed in U.S. Pat. No. 10,392,390, which is incorporated herein by reference in its entirety for all purposes.

CONVULSIVE/MOTOR SEIZURES: As used here, a “convulsive/motor seizure” refers to a tonic-clonic, tonic, tonic-atonic leading to drop, focal motor, epileptic spasms, myoclonic-atonic leading to drop and seizures. Non-convulsive seizures include myoclonic, absence, atypical absence, or atonic seizures and focal seizures without an observable motor component.

CONVULSIVE/MOTOR SEIZURE-FREE DAY: As used herein, a “convulsive/motor seizure-free day” refers to a day for which diary data are available and no convulsive/motor seizures were reported.

DROP SEIZURE: As used herein, the term “drop seizure” refers to a seizure involving the entire body, trunk or head that leads to a fall, injury, slumping in a chair, or head hitting the surface, or could have led to a fall or injury, depending on the position of the subject at the time of the attack or spell.

AGONIST: As used herein, the term “agonist” refers to a moiety that interacts with and activates a receptor, such as the 5-HT_{2C} serotonin receptor, and initiates a physiological or pharmacological response characteristic of that receptor.

ADMINISTERING: As used herein, “administering” means to provide a compound or other therapy, remedy, or treatment such that a patient internalizes a compound.

ORAL or ORALLY: As used herein, “oral” or “orally” refers to administration of a compound or composition to a patient by a route or mode along the alimentary canal. Examples of enteral routes of administration include, without, limitation, oral, as in swallowing solid (e.g., tablet) or liquid (e.g., syrup) forms; sub-lingual (absorption under the tongue); nasojejunal or gastrostomy tubes (into stomach); intraduodenal administration; as well as rectal administration (e.g., suppositories for release and absorption of a compound or composition by in the lower intestinal tract of the alimentary canal).

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to a patient. In this case the health care practitioner may or may not provide a prescription for the compound, dosage regimen, or treatment. Further, the health care practitioner may or may not provide the recommended compound or treatment. For example, the health care practitioner can advise the patient where to obtain the compound without providing the compound. In some embodiments, a health care practitioner can provide a prescription for the compound, dosage regimen, or treatment to the patient. For example, a health care practitioner can give a written or oral prescription to a patient. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the patient. As used herein, giving a sample of a compound constitutes an implicit prescription for the compound. Different health care systems around the world use different methods for prescribing and/or administering compounds or treatments and these methods are encompassed by the disclosure.

A prescription can include, for example, a patient's name and/or identifying information such as date of birth. In addition, for example, a prescription can include: the medication name, medication strength, dosage, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a condition described herein. In addition, a healthcare practitioner can include anyone who can recommend, prescribe, administer, or prevent a patient from receiving a compound or drug including, for example, an insurance provider.

PREVENT, PREVENTING, OR PREVENTION: As used herein, the term “prevent,” “preventing”, or “prevention,” such as prevention of a particular disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to a patient who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein, the term “treat,” “treating”, or “treatment” means the administration of therapy to a patient who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, a patient is said to “tolerate” a dosage of a compound if administration of that dosage to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the patient is not able to tolerate the combination of headache and vomiting, even if the severity of each is less than when experienced alone.

INTOLERANCE: As used herein, “intolerance” means significant toxicities and/or tolerability issues that led to a reduction in dosage or discontinuation of the medication. “Intolerance” can be replaced herein with the term “unable to tolerate.”

ADVERSE EVENT: As used herein, an “adverse event” is an untoward medical occurrence that is associated with treatment with Compound, 1, or a pharmaceutically acceptable salt thereof. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g., physician, nurse, nurse practitioner, etc.) that a patient requires or will benefit from treatment. This judgment is made based on a variety of factors that are in the realm of a caregiver's expertise, but that includes the knowledge that the patient is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

PATIENT: As used herein, “patient” means any human. In some embodiments, a human individual is referred to a “subject,” “participant,” or “individual.”

DOSE: As used herein, “dose” means a quantity of Compound 1, or a pharmaceutically acceptable salt thereof, given to the patient for treating or preventing the disease or disorder at one specific time. As used herein, “dosage” refers to the amount of Compound 1, or a pharmaceutically acceptable salt thereof, in one or more doses.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dosage.

PHARMACEUTICAL COMPOSITION: As used herein, “pharmaceutical composition” means a composition comprising at least one active ingredient, such as Compound 1, including but not limited to, salts of Compound 1, whereby the composition is amenable to investigation for a specified, efficacious outcome. Those of ordinary skill in the art will understand and appreciate the techniques appropriate for determining whether an active ingredient has a desired efficacious outcome based upon the needs of the artisan.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

STEADY STATE: As used herein, “steady-state” is reached when the quantity of a drug eliminated in a given unit of time equals the quantity of the drug that reaches systemic circulation in the unit of time. A “steady-state” pharmacokinetic characteristic means the characteristic can be achieved upon reaching steady-state.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It will be apparent to those skilled in the art that the dosage forms described herein may comprise, as the active component, either Compound 1, or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, DE), Avantium Technologies (Amsterdam) and Aptuit (Greenwich, CT).

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1, or a pharmaceutically acceptable salt thereof can be separated into two methods; one method reciting prescribing Compound 1, or a pharmaceutically acceptable salt thereof and the other method reciting administering Compound 1, or a pharmaceutically acceptable salt thereof. In addition, for example, a method that recites prescribing Compound 1, or a pharmaceutically acceptable salt thereof and a separate method of the invention reciting administering Compound 1, or a pharmaceutically acceptable salt thereof can be combined into a single method reciting prescribing and/or administering Compound 1, or a pharmaceutically acceptable salt thereof.

### METHODS

Provided is a method of treating or preventing a 5-hydroxytryptamine (HT)_{2C} receptor-associated disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof. In some embodiments, Compound 1, or a pharmaceutically acceptable salt thereof, is administered twice daily.

Also provided is a method of treating or preventing epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof. In some embodiments, Compound 1, or a pharmaceutically acceptable salt thereof, is administered twice daily.

In some embodiments, the epilepsy is a focal epilepsy, a generalized epilepsy, or a combined generalized and focal epilepsy. In some embodiments, the epilepsy has one or more of a structural etiology, a genetic etiology, an infectious etiology, a metabolic etiology, and an immune etiology.

Also provided is a method of reducing severity of an epileptic seizure in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof. In some embodiments, Compound 1, or a pharmaceutically acceptable salt thereof, is administered twice daily.

Also provided is a method of reducing the frequency of epileptic seizures in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof. In some embodiments, Compound 1, or a pharmaceutically acceptable salt thereof, is administered twice daily.

In some embodiments, the epileptic seizure is a focal seizure or a generalized seizure. In some embodiments, the epileptic seizure has one or more of a structural etiology, a genetic etiology, an infectious etiology, a metabolic etiology, and an immune etiology.

Also provided is a method of treating or preventing a seizure disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof. In some embodiments, Compound 1, or a pharmaceutically acceptable salt thereof, is administered twice daily.

In some embodiments, the seizure disorder is a focal seizure disorder, a generalized seizure disorder, or combined generalized and focal seizure disorder. In some embodiments, the seizure disorder has one or more of a structural etiology, a genetic etiology, an infectious etiology, a metabolic etiology, and an immune etiology.

In some embodiments, the seizure disorder is selected from epilepsy, epilepsy with generalized tonic-clonic seizures, epilepsy with myoclonic absences, frontal lobe epilepsy, temporal lobe epilepsy, Landau-Kleffner Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, or EIDFEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

Also provided is a method of treating or preventing developmental and epileptic encephalopathy (DEE) in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof. In some embodiments, Compound 1, or a pharmaceutically acceptable salt thereof, is administered twice daily.

In some embodiments, the DEE is chosen from epilepsy syndromes that involve a developmental impairment related to the underlying etiology (e.g., genetic mutation), frequent epileptiform activity, or both. In some embodiments, the DEE is epileptic. Epileptic encephalopathy (EE) can involve frequent epileptiform activity that contributes to an extent of cognitive and behavioral impartments that is more severe than what might be expected based on underlying etiology alone. In some embodiments, the DEE is developmental. Developmental encephalopathy (DE) can involve developmental consequences (e.g., developmental slowing or regression) that arise directly from underlying etiology, and may not involve frequent epileptic activity. In some embodiments, the DEE is both epileptic and developmental, in which developmental and epileptic factors both contribute to encephalopathy.

In some embodiments, the DEE is an early infantile DEE (EIDEE, or Ohtahara syndrome). In some embodiments, the EIDEE is an encephalopathy related to mutations in one or more of ARHGEF9, ARX, BRATI, CASK, CDKL5, CYFIP2, DMXL2, GNAO1, KCNQ2, KCNT1, NECAP1, NSF, PCDH19, PIGA, PLCB1, PNKP, SCN2A, SCN8A, SIK1, SLC13A5, SLC25A22, SPTAN1, ST3GAL3, STXBP1, TBC1D24, WWOX, GLDC, AMT, GCSH, ALDH7A1, and PNPO. In some embodiments, the EIDEE is an encephalopathy associated with nonketotic hyperglycinemia (NKH; e.g., related to mutations in one or more of GLDC, AMT, GCSH), cortical dysplasia, and mitochondrial disorders.

In some embodiments, the EIDEE is a KCNQ2 encephalopathy, an SCN2A encephalopathy, an SCN8A encephalopathy, or a pyridoxine-dependent epilepsy (PDE).

In some embodiments, the DEE is an infantile epileptic spasms syndrome (IESS), which includes infants who present with epileptic spasms who do not fulfill the criteria for West syndrome. In some embodiments, the IESS is an encephalopathy related to mutations in one or more of AARS, ACADS ALG1, ALG13, ALPL, AMT, ARX, ATP2A2, ATP7A, CACNA1A, CACNAlC, CDKL5, CD99L2, CLCN4, CLCN6, CYFIP1, CYFIP2, DCX, DNM1, EEF1A2, FLNA, FOXG1, GABRE, GCSH, GLDC, GNAO1, GNB1, GPT2, GRIN2A, GRIN2B, HCN1, HEXA, IRF2BPL, KCNB1, KCNJ11, KCNQ2, KCNT1, KIF1A, KMT2D, LIS1 (also referred to as PAFAHIB1), MAGI2, MECP2, MED12, MEF2C, MMACHC MT-ND1, MYO18A, NEDD4L, NF1, NPRL3, NTRK2, PNKP, SCN2A, SCN8A, SCN10A, SETBP1, SETD5, SLC25A22, SLC35A2, SMARCA2, SPTAN1, STXBP1, TAF1, TBL1XR1, TCF4, TCF20, TSC1, TSC2, TUBA1A, UFC1, and WDR45. In some embodiments, the EISS is an encephalopathy related to 17p13.3 microdeletion, Xp22.13 microdeletion, 20q13.33 microdeletion, 9q33.3-34.11 microdeletion, 9p24.3-22.3 microdeletion, 5p12-11 microduplication, 3p25.3 microdeletion, 1p³6.33 microdeletion, Xp22.11-21.3 microduplication, or 15q11.2 microduplication.

In some embodiments, the IESS is an encephalopathy associated with chromosome syndromes (e.g., 1p36 deletion syndrome, tetrasomy 12p, dup15q syndrome, trisomy 21 (Down syndrome)), NKH, organic acidemias, hypoxic ischemic encephalopathy (HIE), neurofibromatosis, intracranial infection, brain injury secondary to neonatal hypoglycemia, intracranial hemorrhage (ICH), encephalomalacia, neuroglioma, focal brain lesion, pachygyria-lissencephaly, focal cortical dysplasia, heterotopia, polymicrogyria, schizencephaly, agenesis of the corpus callosum, intracranial hemangioma, Menkes disease, neurodegeneration with brain iron accumulation, methylmalonic acidemia (MMA), short-chain acyl-CoA dehydrogenase (SCAD) deficiency, lysosomal storage diseases, mitochondrial disorders, intra-uterine infection, GLUT-1 deficiency syndrome (hypoglycorrhachia), leukoencephalopathy, and hypophosphatasia.

In some embodiments, the IESS is tuberous sclerosis complex (TSC; associated with mutations in TSC1 or TSC2), an ARX encephalopathy, a CDKL5 encephalopathy (CDK5L deficiency disorder), or an STXBP1 encephalopathy.

In some embodiments, the DEE is chosen from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.

In some embodiments, the DEE is chosen from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

Also provided is a method of treating or preventing a refractory epilepsy in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof, wherein Compound 1, or a pharmaceutically acceptable salt thereof. In some embodiments, Compound 1, or a pharmaceutically acceptable salt thereof, is administered twice daily.

In some embodiments, the patient has a comorbid condition, such as intellectual disability, autism spectrum disorder, and/or behavioral problems.

In some embodiments, the method provides improvement in at least one symptom selected from ataxia, gait impairment, speech impairment, vocalization, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures.

In some embodiments, the administration results in an improvement in the frequency of convulsive/motor seizures and other seizure types. In some embodiments, the administration results in an improvement in one or more of the following:
frequency of observed countable motor seizures;
number of total seizures;
frequency of non-convulsive seizure;
number of episodes of status epilepticus;
frequency of use of rescue medication; and/or
number of countable motor seizure-free days.

In some embodiments, the administration results in an improvement in the Subject/Caregiver and Investigator Clinical Global Impression of Improvement (CGI-I), the Investigator Clinical Global Impression of Severity (CGI-S), and/or the 55-item Quality of Life in Childhood Epilepsy Questionnaire (QOLCE-55). In some embodiments, the administration results in at least a 1-point change from baseline in CGI-I and/or CGI-S.

In some embodiments, prior to administration, the patient had treatment resistant countable motor seizures with an average of ≥4 observed/countable motor seizures per 4-week period while on stable ASM treatment.

In some embodiments, the patient has a DEE but does not have Dravet syndrome or Lennox-Gastaut Syndrome.

In some embodiments wherein the patient has a DEE but does not have Dravet syndrome or Lennox-Gastaut Syndrome, the patient had:
a history of onset of unprovoked seizures at 5 years of age or earlier;
a history of developmental delay;
a history of combined focal and generalized seizure types or multiple generalized seizure types;
a history of slow or disorganized electroencephalogram; and/or
no history of idiopathic generalized seizures.

In some embodiments, the patient has Dravet syndrome.

In some embodiments wherein the patient has Dravet syndrome, prior to administration, the patient had:
onset of seizures between 3 and 12 months of age in an otherwise healthy infant; a history of seizures that were either generalized tonic-clonic or unilateral clonic or bilateral clonic;
normal initial development; and/or
a history of developmental delay.

In some embodiments wherein the patient has Dravet syndrome, prior to administration, the patient had:
an emergence of another seizure type;
prolonged exposure to warm temperatures-induced seizures and/or seizures that were associated with fevers due to illness or vaccines, hot baths, high levels of activity, and sudden temperature changes, and/or
seizures were induced by strong natural and/or fluorescent lighting.

In some embodiments wherein the patient has Dravet syndrome, prior to administration, the patient had genetic test results consistent with a diagnosis of Dravet syndrome.

In some embodiments, the patient has Lennox-Gastaut Syndrome.

In some embodiments wherein the patient has Lennox-Gastaut Syndrome, prior to administration, the patient had:
a history of tonic seizures or tonic/atonic seizures;
more than 1 type of generalized seizure, including but not limited to generalized tonic-clonic, tonic-atonic, atonic, tonic, myoclonic, or drop seizures;
a history of seizure before 8 years of age.
a history of developmental delay.
a previous electroencephalogram reporting diagnostic criteria for Lennox-Gastaut Syndrome (abnormal inter-ictal electroencephalogram background activity accompanied by inter-ictal slow spike-and-wave pattern ≤2.5 hertz or interictal generalized paroxysmal fast activity); and/or
an average of ≥4 observed drop seizures per 4-week while on stable ASM treatment.

In some embodiments, the total daily dosage of Compound 1, or a pharmaceutically acceptable salt thereof, is equivalent to about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 mg of Compound 1. In some embodiments, the total daily dosage of Compound 1, or a pharmaceutically acceptable salt thereof, is equivalent to about, 6, 9, 12, 15, 18, 21, 24, 27, 30, 33, 36, 39, 42, 45, 48, 51, 54, 57, 60, 63, 66, 69, or 72 mg of Compound 1. In some embodiments, the total daily dosage of Compound 1, or a pharmaceutically acceptable salt thereof, is equivalent to about 6 mg of Compound 1. In some embodiments, the total daily dosage of Compound 1, or a pharmaceutically acceptable salt thereof, is equivalent to about 9 mg of Compound 1. In some embodiments, the total daily dosage of Compound 1, or a pharmaceutically acceptable salt thereof, is equivalent to about 12 mg of Compound 1. In some embodiments, the total daily dosage of Compound 1, or a pharmaceutically acceptable salt thereof, is equivalent to about 18 mg of Compound 1. In some embodiments, the total daily dosage of Compound 1, or a pharmaceutically acceptable salt thereof, is equivalent to about 24 mg of Compound 1. In some embodiments, the total daily dosage of Compound 1, or a pharmaceutically acceptable salt thereof, is equivalent to about 30 mg of Compound 1. In some embodiments, the total daily dosage of Compound 1, or a pharmaceutically acceptable salt thereof, is equivalent to about 36 mg of Compound 1. In some embodiments, the total daily dosage of Compound 1, or a pharmaceutically acceptable salt thereof, is equivalent to about 54 mg of Compound 1. In some embodiments, the total daily dosage of Compound 1, or a pharmaceutically acceptable salt thereof, is equivalent to about 72 mg of Compound 1.

In some embodiments, Compound 1, or a pharmaceutically acceptable salt thereof, is administered three times daily in a dosage equivalent to about 3 mg/dose of Compound 1. In some embodiments, the Compound 1, or a pharmaceutically acceptable salt thereof, is administered three times daily in a dosage equivalent to about 6 mg/dose of Compound 1. In some embodiments, Compound 1, or a pharmaceutically acceptable salt thereof, is administered three times daily in a dosage equivalent to about 9 mg/dose of Compound 1. In some embodiments, Compound 1, or a pharmaceutically acceptable salt thereof, is administered three times daily in a dosage equivalent to about 12 mg/dose of Compound 1. In some embodiments, Compound 1, or a pharmaceutically acceptable salt thereof, is administered three times daily in a dosage equivalent to about 18 mg/dose of Compound 1. In some embodiments, Compound 1, or a pharmaceutically acceptable salt thereof, is administered three times daily in a dosage equivalent to about 24 mg/dose of Compound 1.

In some embodiments, Compound 1, or a pharmaceutically acceptable salt thereof, is administered twice daily in a dosage equivalent to about 3 mg/dose of Compound 1. In some embodiments, the Compound 1, or a pharmaceutically acceptable salt thereof, is administered twice daily in a dosage equivalent to about 6 mg/dose of Compound 1. In some embodiments, Compound 1, or a pharmaceutically acceptable salt thereof, is administered twice daily in a dosage equivalent to about 9 mg/dose of Compound 1. In some embodiments, Compound 1, or a pharmaceutically acceptable salt thereof, is administered twice daily in a dosage equivalent to about 12 mg/dose of Compound 1. In some embodiments, Compound 1, or a pharmaceutically acceptable salt thereof, is administered twice daily in a dosage equivalent to about 15 mg/dose of Compound 1. In some embodiments, Compound 1, or a pharmaceutically acceptable salt thereof, is administered twice daily in a dosage equivalent to about 18 mg/dose of Compound 1. In some embodiments, Compound 1, or a pharmaceutically acceptable salt thereof, is administered twice daily in a dosage equivalent to about 24 mg/dose of Compound 1.

In some embodiments, administration of Compound 1 as described herein can achieve one or more pharmacokinetic characteristics, including a geometric mean steady-state C_{trough} in CSF, a geometric mean steady-state C_{trough} in plasma, or both. In some embodiments, the administration results in a ratio of a geometric mean steady-state C_{trough} of Compound 1 in CSF to a geometric mean steady-state C_{trough} of Compound 1 in plasma (CSF/P C_{trough}) of at least about 1.4. In some embodiments, the administration results in a CSF/P C_{trough} of about 1.4 to about 3, about 1.4 to about 2.5, about 1.4 to about 2, about 1.5 to about 3, about 1.5 to about 2.5, or about 1.5 to about 2. In some embodiments, the administration results in a CSF/P C_{trough} of about 1.5, about 1.7, or about 1.9.

In some embodiments, the administration results in a geometric mean steady-state C_{trough} of Compound 1 in CSF of about 3 ng/mL to about 15 ng/mL, for example, about 3 ng/mL to about 12 ng/mL, about 3 ng/mL to about 10 ng/mL, about 4 ng/mL to about 15 ng/mL, about 4 ng/mL to about 12 nm/mL, about 4 ng/mL to about 10 ng/mL, about 5 ng/mL to about 15 ng/mL, about 5 ng/mL to about 12 ng/mL, about 5 ng/L to about 10 ng/mL. In some embodiments, the administration results in a geometric mean steady-state C_{trough} of Compound 1 in CSF of about 6.5 ng/mL, about 7 ng/mL, or about 7.7 ng/mL.

In some embodiments, the administration results in a geometric mean steady-state C_{trough} of Compound 1 in plasma of about 2 ng/mL to about 12 ng/mL, for example, about 2 ng/mL to about 10 ng/mL, about 2 ng/mL to about 9 ng/mL, about 3 ng/mL to about 12 ng/mL, about 3 ng/mL to about 10 ng/mL, about 3 ng/mL to about 9 ng/mL, about 4 ng/mL to about 12 ng/mL, about 4 ng/mL to about 10 ng/mL, or about 4 ng/mL to about 9 ng/mL. In some embodiments, the administration results in a geometric mean steady-state C_{trough} of Compound 1 in plasma of about 4.0 ng/mL, about 4.4 ng/mL, about 4.9 ng/mL, about 5.2 ng/mL, about 5.6 ng/mL, about 6.5 ng/mL, about 7.1 ng/mL, or about 7.4 ng/mL.

In some embodiments, Compound 1, or a pharmaceutically acceptable salt thereof, is administered via a titration scheme that includes the up-titration of Compound 1, or a pharmaceutically acceptable salt thereof, until an optimized dosage is administered.

In some embodiments, the total daily dosage of Compound 1, or a pharmaceutically acceptable salt thereof, is titrated to a dosage equivalent to about 9 mg of Compound 1. In some embodiments, the total daily dosage of Compound 1, or a pharmaceutically acceptable salt thereof, is titrated to a dosage equivalent to about 18 mg of Compound 1. In some embodiments, the total daily dosage of Compound 1, or a pharmaceutically acceptable salt thereof, is titrated to a dosage equivalent to about 24 mg of Compound 1. In some embodiments, the total daily dosage of Compound 1, or a pharmaceutically acceptable salt thereof, is titrated to a dosage equivalent to about 30 mg of Compound 1. In some embodiments, the total daily dosage of Compound 1, or a pharmaceutically acceptable salt thereof, is titrated to a dosage equivalent to about 36 mg of Compound 1. In some embodiments, the total daily dosage of Compound 1, or a pharmaceutically acceptable salt thereof, is titrated to a dosage equivalent to about 54 mg of Compound 1. In some embodiments, the total daily dosage of Compound 1, or a pharmaceutically acceptable salt thereof, is titrated to a dosage equivalent to about 72 mg of Compound 1.

In some embodiments, the total daily dosage of Compound 1, or a pharmaceutically acceptable salt thereof, is down-titrated to a dosage equivalent to about, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 mg of Compound 1. In some embodiments, the total daily dosage of Compound 1, or a pharmaceutically acceptable salt thereof, is down-titrated to a dosage equivalent to about 54 mg of Compound 1. In some embodiments, the total daily dosage of Compound 1, or a pharmaceutically acceptable salt thereof, is down-titrated to a dosage equivalent to about 36 mg of Compound 1. In some embodiments, the total daily dosage of Compound 1, or a pharmaceutically acceptable salt thereof, is down-titrated to a dosage equivalent to about 30 mg of Compound 1. In some embodiments, the total daily dosage of Compound 1, or a pharmaceutically acceptable salt thereof, is down-titrated to a dosage equivalent to about 24 mg of Compound 1. In some embodiments, the total daily dosage of Compound 1, or a pharmaceutically acceptable salt thereof, is down-titrated to a dosage equivalent to about 18 mg of Compound 1. In some embodiments, the total daily dosage of Compound 1, or a pharmaceutically acceptable salt thereof, is down-titrated to a dosage equivalent to about 12 mg of Compound 1. In some embodiments, the total daily dosage of Compound 1, or a pharmaceutically acceptable salt thereof, is down-titrated to a dosage equivalent to about 9 mg of Compound 1. In some embodiments, the total daily dosage of Compound 1, or a pharmaceutically acceptable salt thereof, is down-titrated to a dosage equivalent to about 6 mg of Compound 1. In some embodiments, the total daily dosage of Compound 1, or a pharmaceutically acceptable salt thereof, is down-titrated to a dosage equivalent to about 3 mg of Compound 1.

In some embodiments, the increasing of the dosage of Compound 1, or a pharmaceutically acceptable salt thereof, in increments equivalent to about 1.5 mg/dose of Compound 1 about every 1, 2, 3, 4, 5, 6, 7, 8, 9, or 10 days until the optimized dosage is administered. In some embodiments, the increasing of the dosage of Compound 1, or a pharmaceutically acceptable salt thereof, in increments equivalent to about 3 mg/dose of Compound 1 about every 1, 2, 3, 4, 5, 6, 7, 8, 9, or 10 days until the optimized dosage is administered. In some embodiments, the optimized dosage is equivalent to about 3 mg/dose of Compound 1. In some embodiments, the optimized dosage is equivalent to about 6 mg/dose of Compound 1. In some embodiments, the optimized dosage is equivalent to about 9 mg/dose of Compound 1. In some embodiments, the optimized dosage is equivalent to about 12 mg/dose of Compound 1. In some embodiments, the optimized dosage is equivalent to about 15 mg/dose of Compound 1. In some embodiments, the optimized dosage is equivalent to about 18 mg/dose of Compound 1.

In some embodiments, the titration scheme comprises prescribing and/or administering Compound 1, or a pharmaceutically acceptable salt thereof, at an initial dosage (also referred to as a starting dosage) equivalent to about 1 mg/dose, about 2 mg/dose, about 3 mg/dose, about 4 mg/dose, about 5 mg/dose, or about 6 mg/dose of Compound 1 and, provided that the patient tolerates the initial dosage, increasing the dosage. In some embodiments, the titration scheme comprises prescribing and/or administering Compound 1, or a pharmaceutically acceptable salt thereof, at an initial dosage equivalent to about 1 mg/dose, about 2 mg/dose, about 3 mg/dose, about 4 mg/dose, about 5 mg/dose, or about 6 mg/dose of Compound 1 and, provided that the patient tolerates the initial dosage and that the patient has not had an adequate response, increasing the dosage. In some embodiments, the titration scheme comprises prescribing and/or administering Compound 1, or a pharmaceutically acceptable salt thereof, two or three times daily at an initial dosage equivalent to about 1 mg/dose, about 2 mg/dose, about 3 mg/dose, about 4 mg/dose, about 5 mg/dose, or about 6 mg/dose of Compound 1 for about 1, 2, 3, 4, 5, 6, 7, 8, 9, or 10 days and, provided that the patient tolerates the initial dosage, increasing the dosage. In some embodiments, the titration scheme comprises prescribing and/or administering Compound 1, or a pharmaceutically acceptable salt thereof, two or three times daily at an initial dosage equivalent to about 1 mg/dose, about 2 mg/dose, about 3 mg/dose, about 4 mg/dose, about 5 mg/dose, or about 6 mg/dose of Compound 1 for about 1, 2, 3, 4, 5, 6, 7, 8, 9, or 10 days and, provided that the patient tolerates the initial dosage and that the patient has not had an adequate response, increasing the dosage. In some embodiments, the titration scheme comprises prescribing and/or administering Compound 1, or a pharmaceutically acceptable salt thereof, at an initial dosage equivalent to about 6 mg/dose of Compound 1 for about 2 days and, provided that the patient tolerates the initial dosage and that the patient has not had an adequate response, increasing the dosage.

In some embodiments, the increased dosage is optimized for further response. In some embodiments, the increased dosage is equivalent to about 3 mg/dose of Compound 1. In some embodiments, the increased dosage is equivalent to about 6 mg/dose of Compound 1. In some embodiments, the increased dosage is equivalent to about 9 mg/dose of Compound 1. In some embodiments, the increased dosage is equivalent to about 12 mg/dose of Compound 1. In some embodiments, the increased dosage is equivalent to about 15 mg/dose of Compound 1. In some embodiments, the increased dosage is equivalent to about 18 mg/dose of Compound 1.

In some embodiments, the titration scheme further comprises administering Compound 1, or a pharmaceutically acceptable salt thereof, at the increased dosage for about 2, 3, 4, 5, 6, 7, 8, 9, or 10 days. In some embodiments, the titration scheme further comprises administering Compound 1, or a pharmaceutically acceptable salt thereof, at the increased dosage for about two days. In some embodiments, the titration scheme further comprises administering Compound 1, or a pharmaceutically acceptable salt thereof, at the increased dosage for about five days.

In some embodiments, if the patient does not tolerate the increased dosage, the optimized dosage is the initial dosage.

In some embodiments, if the patient tolerates the increased dosage, the optimized dosage is the increased dosage. In some embodiments, if the patient tolerates the increased dosage and if the patient has had an adequate response, the optimized dosage is the increased dosage.

In some embodiments, the titration scheme comprises further increasing the dosage, provided that the individual tolerates the increased dosage. In some embodiments, the titration scheme comprises further increasing the dosage, provided that the individual tolerates the increased dosage and that the individual has not had an adequate response. In some embodiments, the further increased dosage is optimized for further response. In some embodiments, the further increased dosage is equivalent to about 12 mg/dose of Compound 1. In some embodiments, the further increased dosage is equivalent to about 15 mg/dose of Compound 1. In some embodiments, the further increased dosage is equivalent to about 18 mg/dose of Compound 1.

In some embodiments, the titration scheme further comprises administering Compound 1, or a pharmaceutically acceptable salt thereof, at the further increased dosage for about 2, 3, 4, 5, 6, 7, 8, 9, or 10 days. In some embodiments, the titratrion scheme further comprises administering Compound 1, or a pharmaceutically acceptable salt thereof, at the further increased dosage for about two days. In some embodiments, the titration scheme further comprises administering Compound 1, or a pharmaceutically acceptable salt thereof, at the further increased dosage for about five days.

In some embodiments, if the patient does not tolerate the further increased dosage, the optimized dosage is the increased dosage.

In some embodiments, if the patient tolerates the further increased dosage, the optimized dosage is the further increased dosage. In some embodiments, if the patient tolerates the further increased dosage and if the patient has had an adequate response, the optimized dosage is the further increased dosage.

In some embodiments, the titration scheme further comprises administering the optimized dosage of Compound 1, or a pharmaceutically acceptable salt thereof, to the patient.

In some embodiments, if the patient tolerates the increased dosage and if the patient has not had an adequate response, the method further comprises increasing the dosage.

In some embodiments, the titration scheme further comprises administering the optimized dosage of Compound 1, or a pharmaceutically acceptable salt thereof, to the patient.

In some embodiments, for example, when the patient does not tolerate the increased dosage of Compound 1, or a pharmaceutically acceptable salt thereof, the titration scheme further comprises down-titration of Compound 1, or a pharmaceutically acceptable salt thereof. In some embodiments, the down-titration comprises reducing the dosage of Compound 1, or a pharmaceutically acceptable salt thereof, being administered to the patient by an increment equivalent to about, 1, 2, 3, 3, 4, 5, 6, 7, 8, 9, 10, 11, or 12 mg/dose of Compound 1. In some embodiments, the down-titration scheme comprises reducing the dosage of Compound 1, or a pharmaceutically acceptable salt thereof, being administered to the patient once. In some embodiments, the down-titration scheme comprises reducing the dosage of Compound 1, or a pharmaceutically acceptable salt thereof, being administered to the patient more than once. In some embodiments, the down-titration scheme comprises reducing the dosage of Compound 1, or a pharmaceutically acceptable salt thereof, being administered to the patient in increments equivalent to about 3 mg/dose of Compound 1 about every 1, 2, 3, 4, or 5 days until the patient is no longer being administered Compound 1, or a pharmaceutically acceptable salt thereof.

In some embodiments, the dosage of Compound 1, or a pharmaceutically acceptable salt thereof, is down-titrated to address an observed side effect. In some embodiments, the dosage of Compound 1, or a pharmaceutically acceptable salt thereof, is down-titrated to minimize the risk of a withdrawal induced side effect.

In some embodiments, the patient is also being administered an antiepileptic drug or antiseizure medicine. In some embodiments, the patient is also being administered an antiepileptic drug effective in suppressing interictal epileptiform discharges (e.g., benzodiazepines, valproic acid, and lamotrigine). In some embodiments, the patient is also being administered an immunomodulatory therapy (e.g., corticosteroids, intravenous immunoglobulin [IVIG], plasmapheresis). In some embodiments, the patient is also being administered a ketogenic diet. In some embodiments, the patient is also being administered vagal nerve stimulation (VNS) or deep brain stimulation (DBS).

In some embodiments, the patient is also being administered a CYP enzyme inhibitor, and no adjustments to the dosage of Compound 1, or a pharmaceutically acceptable salt thereof, are made as compared to a corresponding patient not being administered the CYP enzyme inhibitor. In some embodiments, the CYP enzyme inhibitor is a moderate CYP enzyme inhibitor or a strong CYP enzyme inhibitor. In some embodiments, the CYXP enzyme inhibitor is an antiseizure medicine. In some embodiments, the CYP enzyme inhibitor is fenfluramine, carbamazepine, clobazam, cannabidiol, felbamate, phenobarbital, or phenytoin. In some embodiments, the CYP enzyme inhibitor is a substrate for CYP2D6, CYP3A4, CYP2C19, or CYP2C9.

In some embodiments, the patient is also being administered a P-glycoprotein (P-gp) inhibitor, and no dose adjustments to the dosage of Compound 1, or a pharmaceutically acceptable salt thereof, are made as compared to a corresponding patient not being administered the P-gp inhibitor. In some embodiments, the P-gp inhibitor is a P-gp-mediated efflux or renal transporter.

In some embodiments, Compound 1, or a pharmaceutically acceptable salt thereof, is administered as a raw or pure chemical, for example as a powder in capsule formulation.

In some embodiments, Compound 1, or a pharmaceutically acceptable salt thereof, is formulated as a pharmaceutical composition further comprising one or more pharmaceutically acceptable carriers.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

In some embodiments, the Compound 1, or a pharmaceutically acceptable salt thereof, is formulated in a manner suitable for oral administration.

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary. Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For oral administration, the pharmaceutical composition may be in the form of suitable for administration via gastrostomy tube or percutaneous endoscopic gastrostomy tube.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Further embodiments include the embodiments disclosed in the following Examples, which is not to be construed as limiting in any way.

### EXAMPLES

### Example 1: Multiple-Dose Study in Healthy Adult Subjects

A Phase 1, open-label, 4-cohort, multiple-dose study in healthy adult male and female subjects was conducted for Cohorts 1 and 2. Approximately, 20 healthy subjects ages 18 to 55 years and having body mass index of 18.5-30.0 kg/m² (sex balanced in each cohort) were enrolled to ensure at least 6 subjects completed qEEG (quantitative electroencephalogram) and CSF (cerebrospinal fluid) PK (pharmacokinetic) assessments in each cohort.

Subjects were enrolled to 1 of 2 dosage regimens. Subject enrollment was stratified by sex to ensure approximate sex balance in each cohort. All dosage regimens included up-titration and taper-down phases. Compound 1 was administered according to the dosage regimens of Table 1:

TABLE 1
Schedule of Dosing

| Cohort | Dose # | D1 | D2 | D3 | D4 | D5 | D6 | D7 | D8 |
|---|---|---|---|---|---|---|---|---|---|
| 1 | 1 | 3 | 3 | 6 | 6 | 6 | 6 | 6 | 6 |
| (6 mg TID) | 2 | 3 | 3 | 6 | 6 | 6 | 6 | 6 | 6 |
| | 3 | 3 | 3 | 6 | 6 | 6 | 6 | 6 | 6 |
| 2 | 1 | 6 | 6 | 12 | 12 | 12 | 12 | 12 | 12 |
| (12 mg TID) | 2 | 6 | 6 | 12 | 12 | 12 | 12 | 12 | 12 |
| | 3 | 6 | 6 | 12 | 12 | 12 | 12 | 12 | 12 |

| Cohort | Dose # | D9 | D10 | D11 | D12 | D13 | D14 | D15 | D16 |
|---|---|---|---|---|---|---|---|---|---|
| 1 | 1 | 6 | 6 | 6 | 6 | 3 | 3 | 3 | 3 |
| (6 mg | 2 | 6 | 6 | — | — | — | — | — | — |
| TID) | 3 | 6 | 6 | 6 | 6 | 3 | 3 | — | — |
| 2 | 1 | 12 | 12 | 12 | 12 | 6 | 6 | 3 | 3 |
| (12 mg | 2 | 12 | 12 | — | — | — | — | — | — |
| TID) | 3 | 12 | 12 | 12 | 6 | 6 | 3 | — | — |

D = Day; D1-D2: up-titration; D3-D11 AM dose (target treatment); taper begins on Day 11 after morning dose.

Subjects received all Compound 1 doses as a liquid formulation (Table 2, below) for oral administration immediately followed by approximately 240 mL of water.

TABLE 2
Compound 1 Liquid Formulation

| | | | Quantity |
|---|---|---|---|
| | | | per |
| | Quality | | Unit |
| Component | Standard | Function | (g/L) |
| Compound 1 HCl | In house | Active | 3.37^{a} |
| KLEPTOSE ® | USP, EP | Taste masking | 100.00 |
| LINECAPS | | | |
| 17 Pea maltodextrin | | | |
| Glycerin, Synthetic | USP, EP, BP, JP | Thickening agent | 25.00 |
| Sucralose | NF | Sweetener | 10.00 |
| Sodium Benzoate | NF | Preservative | 1.80 |
| Methylparaben | NF | Preservative | 1.30 |
| Cherry Blend FX 770 SD | Vendor CoA | Flavorant | 4.50 |
| FD and C Red No. 40 | Vendor CoA | Colorant | 0.05 |
| Diluted HCl, 10% (w/v) | NF | pH adjustment | 2.28 |
| Purified Water | USP | Solvent, diluent | 897.70 |

EP = European Pharmacopeia (Ph. Eur.);
USP = United States Pharmacopeia;
BP = British Pharmacopeia;
JP = Japanese Pharmacopoeia;
CoA = Certificate of Analysis
Quantity of Compound 1 HCl salt required to provide 3 mg of Compound 1 free base

Morning doses were administered following a standard light breakfast (e.g., milk and cereal, toast, and fruit) on all days. TID dosing (three times a day) was approximately 8 hours apart. Subjects fasted from midnight onwards, until the morning breakfast each day. There was no fluid restriction during the study, but subjects should not have consumed excessive amounts of fluid on any day.

For Cohorts 1 and 2, the study consisted of a Screening period (Day −28 to −2), an on-site period from Day −1 to Day 16 and a 10-day postdosing follow-up visit (Day 25). Subjects were admitted to the clinic on Day −1 of the assessment period and remained confined to the clinic until completion of the assessment period (or longer if the Investigator deemed it is clinically indicated for safety follow-up). Safety was assessed continuously from signing of informed consent form through follow up visit. Safety assessments including cardiac troponins, vital signs, 12-lead ECGs (electrocardiograms) were performed at screening and daily from Day −1 to Day 16, and follow-up. C-SSRS (Columbia-suicide severity rating scale) was assessed at screening, Days −1, 11, 16, and 25.

Study assessments related to PK and PD (pharmacodynamics) endpoints were as follows:
Serial CSF samples: Day 11
Serial sampling for plasma protein binding: Day 11
Serial EEGs: Day 1, 3, and Day 10
Trough qEEG: Day 16
Serial serum prolactin samples: Days 1 and 10
Serial plasma PK samples: Days 1, 3, 10, and 11
Trough plasma PK samples: Days 2, 4-9, and 12-16
Bladder ultrasounds were performed on Day −1, 1, 8, and 9
Sampling for pharmacogenomics: Day 1

### Pharmacokinetics

Pharmacokinetic parameters for Compound 1 and its metabolites were determined from the CSF and plasma concentration-time profiles for all evaluable subjects. The parameters are set forth in Table 3, below.

TABLE 3
Pharmacokinetic Parameter Definitions

| Parameter | Definition |
|---|---|
| Cₘₐₓ | Maximum observed concentration in a profile |
| Tₘₐₓ | Time to first occurrence of maximum observed |
| | concentration in a profile |
| C_{trough} | Pre-morning dose concentration |
| AUCₜₐᵤ | Area under the concentration-time curve over |
| | the dosing interval tau |

CSF to free drug plasma ratios of Cₘₐₓ, Tₘₐₓ, and AUCₜₐᵤ were also determined for Compound 1.

Plasma and CSF concentration-time data were tabulated by cohort and timepoint for all subjects, and summarized using descriptive statistics (n, arithmetic mean, SD, median, and range [minimum−maximum]). Mean (SD) and individual concentration-time profiles for plasma and CSF PK were plotted by cohort.

Plasma and CSF PK parameters were estimated using actual dosing and sampling times using non-compartmental methods. Data were tabulated by cohort and subject, and summarized using descriptive statistics (n, arithmetic mean, SD, coefficient of variation [% CV] for arithmetic mean, median, geometric mean, and range [minimum−maximum]. PK parameters were plotted using box-plots or scatter and mean plots to evaluate relationship to dose/dosing regimen.

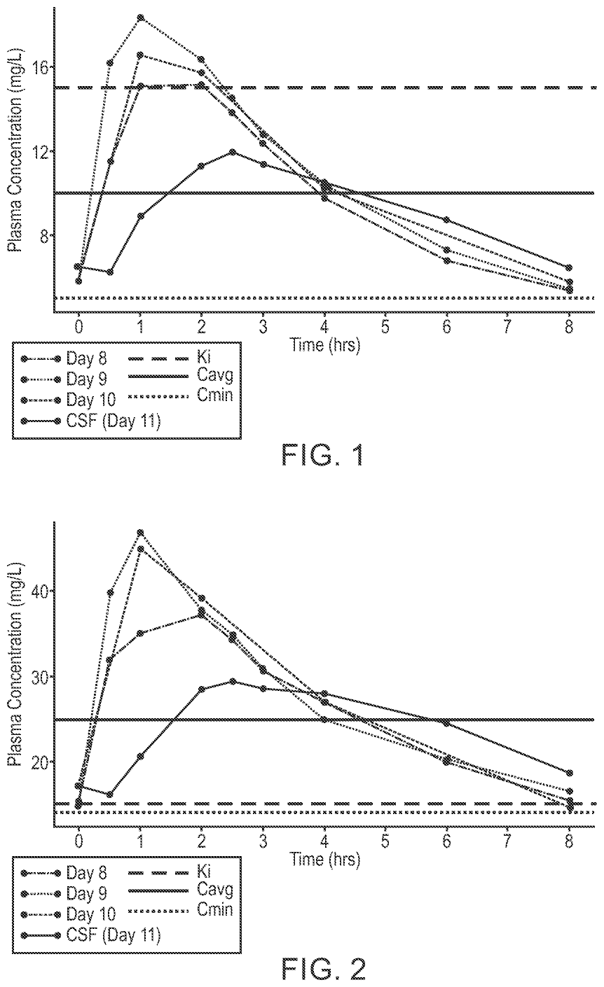

Representative Compound 1 plasma and CSF profiles of Cohort 1 (6 mg TID) and Cohort 2 (12 mg TID) at steady-state are shown in FIGS. 1 and 2, respectively. The relevant inhibition constant (Kᵢ) for 5-HT_{2C} activity is also indicated in FIGS. 1 and 2. As shown in FIG. 2, a majority of participants in Cohort 2 achieved plasma and CSF levels above the relevant Kᵢ throughout the dosing period.

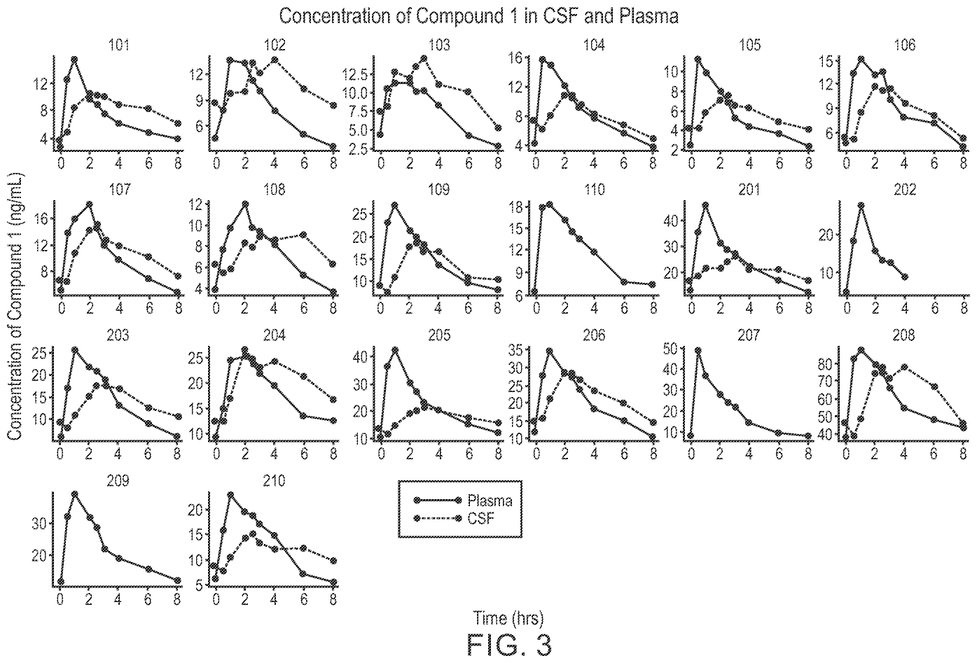

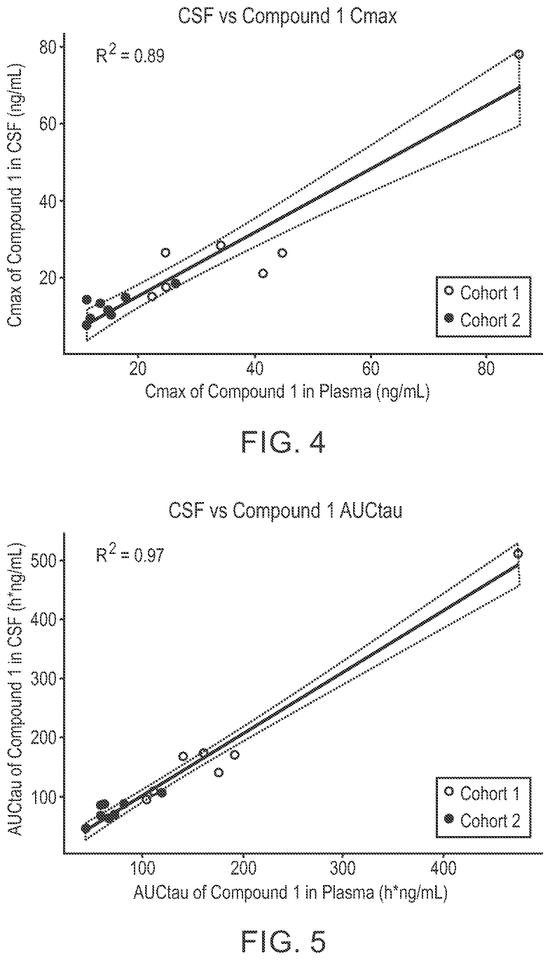

FIG. 3 shows Compound 1 CSF and plasma profiles for individual subjects within Cohort 1 (subjects 101-110) and Cohort 2 (subjects 201-210). Plots comparing Compound 1 Cₘₐₓ and AUCₜₐᵤ parameters in plasma and CSF, shown in FIGS. 4 (Cₘₐₓ) and 5 (AUCₜₐᵤ), indicated strong correlation between plasma and CSF PK parameters.

### Pharmacodynamics

After eligibility assessment, a screening EEG was performed, as part of the screening assessment to exclude subjects with clinically significant abnormalities. Screening EEGs were 20 minutes in duration and included hyperventilation and photic stimulation. They were scored as either within normal limits (0), or as abnormal and exclusionary (1).

All qEEG assessments were performed by registered EEG technicians using individually applied gold cup electrodes and state-of-the-art EEG recording systems. All tests were performed with the participant seated comfortably in a sound-attenuated room. In addition to the standard 19 EEG leads of the International 10/20 system, the left earlobe and right ear lobe were recorded as active leads, and the vertical and horizontal electro-oculograms were recorded as bipolar pairs. The reference electrodes were amplifier-dependent but were usually placed over bony scalp areas near the FCz or FC3 electrode positions.

A five-minute resting qEEG with eyes closed (EC) and a five-minute resting EEG with eyes open (EO) was performed with the participant seated comfortably in a sound-attenuated room. The participant viewed a fixation cross on a video monitor during the eyes-open periods and was instructed not to move their eyes or blink excessively. The resting qEEG was evaluated by means of spectral and coherence analysis, including spectral amplitudes and coherences in clinical frequency bands (delta, theta, alpha1, alpha2, beta1, beta2, beta2, beta3, high beta, gamma) and derived frequency measures (theta-beta ratio (TBR), beta-alpha ratio (BAR), alpha-slow wave index (ASI), dominant frequency, 1/f slope). qEEG was measured within 45 minutes of the planned dose time and again, 1, 2, 4, and 8-hours post-morning dose.

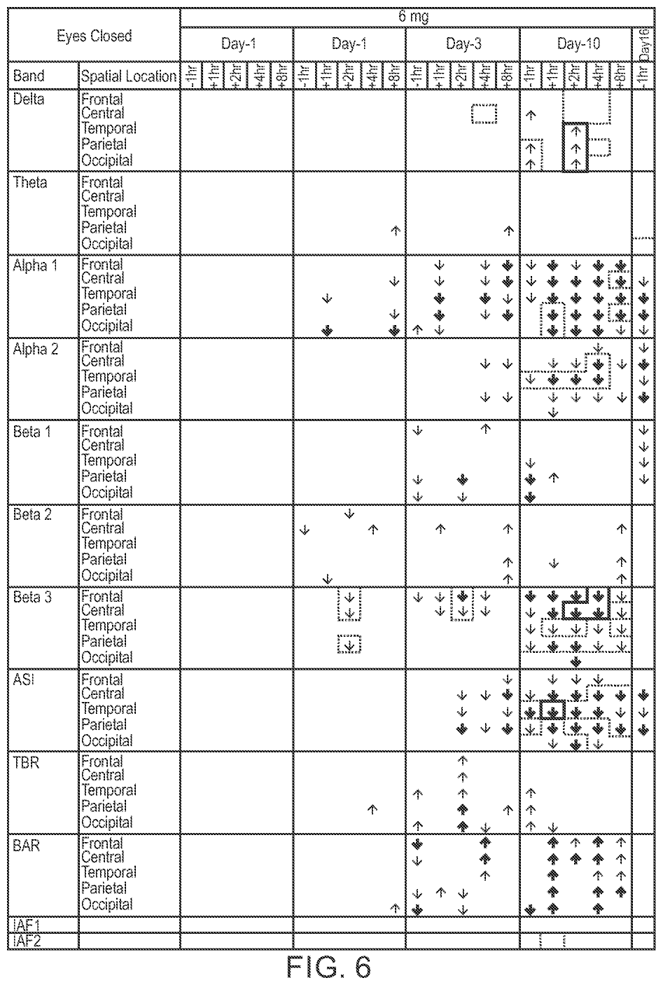

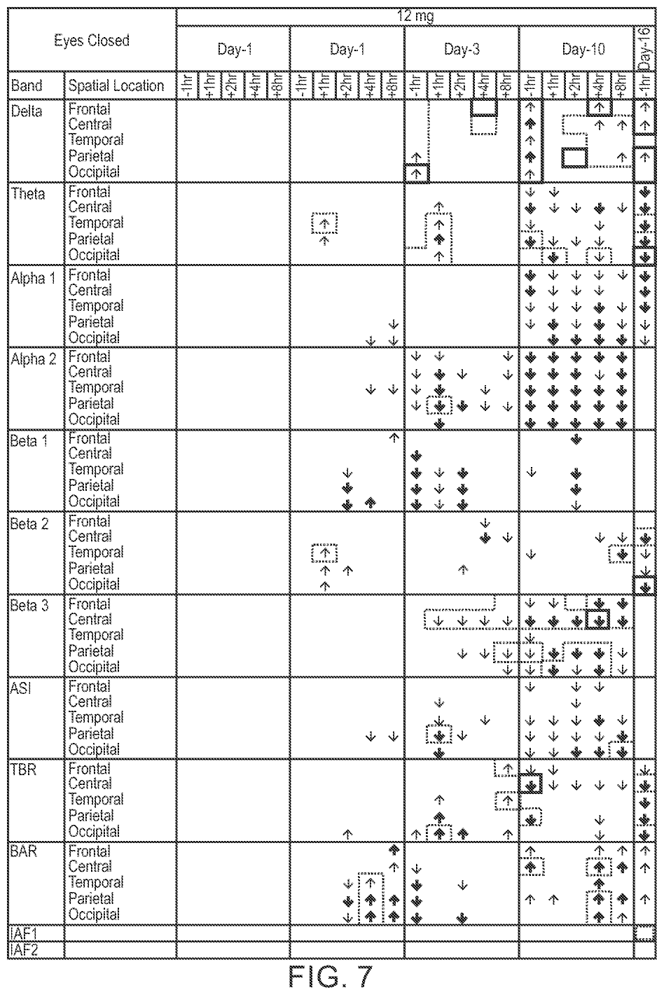

Representative qEEG results for Cohort 1 are shown in FIG. 6, and representative qEEG results for Cohort 2 are shown in FIG. 7. FIGS. 6 and 7 show changes in EC oscillatory band parameters detected by qEEG. Small and large salient contrasts (≥10%, ≥15%) are indicated by light and bold arrows (down=decrease, up=increase) respectively. Small and large salient Cohen's d values (≥0.5, ≥0.8) are indicated by dashed and solid boxes, respectively. Days and timepoints are arranged in columns and bands. The qEEG results of Cohorts 1 and 2, including the results shown in FIGS. 6 and 7, showed progressively (with multiple dosing) and markedly increased EC and EO delta band amplitudes. In Cohort 2, markedly decreased EC and EO theta band amplitudes at Days 10 and 16, and a transient EC theta increase at Day 3, was observed. In Cohorts 1 and 2, progressively and markedly decreased EC alpha1 band amplitudes were observed.

Progressively and markedly decreased EC (Cohorts 1 and 2) and EO (Cohort 2) alpha2 band amplitudes were observed, with certain corresponding decreases in EO alpha2 band amplitudes observed for Cohort 1. Cohort 1 showed transiently increased EO alpha2 and beta1 band amplitudes after dosing, and Cohort 2 showed transiently increased late (8 h) EO alpha1 band amplitudes after dosing.

Cohorts 1 and 2 each showed progressively and markedly decreased EO beta3 band amplitudes, as well as progressive increases in EC and EO BAR, which partly mirrored the decreases seen in the alpha bands. Transiently decreased occipital EO TBR was observed in Cohort 1, and transiently decreased frontal TBR was observed in Cohort 2. These decreases directly mirrored the transient increase in EO beta amplitudes in Cohort 1, and the late (Day 10) increase in beta3 band amplitudes and decrease in theta band amplitudes in Cohort 2.

The outstanding feature of the fractal changes observed by qEEG for Cohorts 1 and 2 was an early (D1-3, +1-4 h) increase in EC gamma band amplitudes, which converted to a late (D10, −1 h/+8 h) decrease with continued dosing. This increase/decrease was corroborated by parallel decreases in TGR and 1/f slope at the same time and regions. The results demonstrated early effects on EEG activity within the first 3 doses, as well as sustained, dose-dependent effects on EEG activity after continuous dosing, indicating receptor engagement.

Pharmacodynamic parameters estimated from EEG and ultrasound were tabulated by subject, cohort, and timepoint. Absolute and percent change from baseline were calculated, with baseline value defined as the last estimate prior to first dose of study drug. All parameters were summarized using descriptive statistics (n, arithmetic mean, SD, median, and range [minimum−maximum]).

The results of Cohorts 1 and 2 showed that plasma and CSF concentration of Compound 1 increased in a dose-dependent and continuous matter, and suggested that Compound 1 engaged neurotransmitter systems and altered the EEG spectrum of subjects. Favorable safety and tolerability results were also observed in Cohorts 1 and 2.

### Example 2: Multiple-Dose Study in Healthy Adult Subjects

The Phase 1, open-label, 4-cohort, multiple-dose study of Example 1 is conducted for Cohorts 3 and 4.

20 further subjects are enrolled to 1 of 2 dosage regimens. Subject enrollment is stratified by sex to ensure approximate sex balance in each cohort. All dosage regimens include up-titration and taper-down phases. Compound 1 is administered according to the dosage regimens of Table 4:

TABLE 4
Schedule of Dosing

| Cohort | Dose # | D1 | D2 | D3 | D4 | D5 | D6 | D7 | D8 |
|---|---|---|---|---|---|---|---|---|---|
| 3 | 1 | 6 | 6 | 12 | 12 | 12 | 12 | 12 | 12 |
| (12 mg BID) | 2 | 6 | 6 | 12 | 12 | 12 | 12 | 12 | 12 |
| 4 | 1 | 9 | 9 | 18 | 18 | 18 | 18 | 18 | 18 |
| (18 mg BID) | 2 | 9 | 9 | 18 | 18 | 18 | 18 | 18 | 18 |

| Cohort | Dose # | D9 | D10 | D11 | D12 | D13 | D14 | D15 | D16 |
|---|---|---|---|---|---|---|---|---|---|
| 3 | 1 | 12 | 12 | 12 | 6 | 3 | 3 | 3 | 3 |
| (12 mg | 2 | 12 | 12 | 6 | 6 | 3 | 3 | — | — |
| BID) | | | | | | | | | |
| 4 | 1 | 18 | 18 | 18 | 9 | 6 | 6 | 6 | 6 |
| (18 mg | 2 | 18 | 18 | 9 | 9 | 6 | 6 | — | — |
| BID) | | | | | | | | | |

Note: D = day; D1-D2: up-titration; D3-D11 AM dose (target treatment); taper begins on Day 11 after morning dose.

Subjects receive all Compound 1 doses as a liquid formulation (Table 2, above) for oral administration immediately followed by approximately 240 mL of water. Morning doses are administered following a standard light breakfast (e.g., milk and cereal, toast, and fruit) on all days. BID dosing (twice a day) is 12 hours apart. Subjects fast from midnight onwards, until the morning breakfast each day. There is no fluid restriction during the study, but subjects should not consume excessive amounts of fluid on any day.

For Cohorts 3 and 4, the study consists of a Screening period (Day −28 to −2), an on-site period from Day −1 to Day 16 and a 10-day post-dosing follow-up visit (Day 25). Subjects are admitted to the clinic on Day −1 of the assessment period and remain confined to the clinic until completion of the assessment period (or longer if the Investigator deems it is clinically indicated for safety follow-up). Safety is assessed continuously from signing of informed consent form through follow up visit. Safety assessments including cardiac troponins, vital signs, 12-lead ECGs (electrocardiograms) are performed at screening and daily from Day −1 to Day 16, and follow-up. C-SSRS (Columbia-suicide severity rating scale) is assessed at screening, Days −1, 11, 16, and 25.

Study assessments related to PK and PD (pharmacodynamics) endpoints are as follows:
Serial CSF samples: Day 11
Serial sampling for plasma protein binding: Day 11
Serial EEGs: Day 1, 3, and Day 10
Trough qEEG: Day 16
Serial serum prolactin samples: Days 1 and 10
Serial plasma PK samples: Days 1, 3, 10, and 11
Trough plasma PK samples: Days 2, 4-9, and 12-16
Bladder ultrasounds are performed on Day −1, 1, 8, and 9
Sampling for pharmacogenomics: Day 1

### Pharmacokinetics

Pharmacokinetic parameters for Compound 1 and its metabolites are determined from the CSF and plasma concentration-time profiles for all evaluable subjects. The parameters are set forth in Table 5, below.

TABLE 5
Pharmacokinetic Parameter Definitions

| Parameter | Definition |
|---|---|
| Cₘₐₓ | Maximum observed concentration in a profile |
| Tₘₐₓ | Time to first occurrence of maximum observed |
| | concentration in a profile |
| C_{trough} | Pre-morning dose concentration |
| AUCₜₐᵤ | Area under the concentration-time curve over |
| | the dosing interval tau. |

CSF to free drug plasma ratios of Cₘₐₓ, Tₘₐₓ, and AUCₜₐᵤ are also determined for Compound 1.

Plasma and CSF concentration-time data are tabulated by cohort and timepoint for all subjects, and summarized using descriptive statistics (n, arithmetic mean, SD, median, and range [minimum−maximum]). Mean (SD) and individual concentration-time profiles for plasma and CSF PK are plotted by cohort.

Plasma and CSF PK parameters are estimated using actual dosing and sampling times using non-compartmental methods. Data are tabulated by cohort and subject, and summarized using descriptive statistics (n, arithmetic mean, SD, coefficient of variation [% CV] for arithmetic mean, median, geometric mean, and range [minimum−maximum]. PK parameters are plotted using box-plots or scatter and mean plots to evaluate relationship to dose/dosing regimen.

### Pharmacodynamics

Prolactin serum concentrations are tabulated by subject, cohort and timepoint. Relationships between plasma and CSF exposure of Compound 1 and metabolites and prolactin are plotted.

After eligibility assessment, a screening EEG is performed, as part of the screening assessment to exclude subjects with clinically significant abnormalities. Screening EEGs are 20 minutes in duration and include hyperventilation and photic stimulation. They are scored as either within normal limits (0), or as abnormal and exclusionary (1).

All qEEG assessments are performed by registered EEG technicians using individually applied gold cup electrodes and state-of-the-art EEG recording systems. All tests are performed with the participant seated comfortably in a sound-attenuated room. In addition to the standard 19 EEG leads of the International 10/20 system, the left earlobe and right ear lobe are recorded as active leads, and the vertical and horizontal electro-oculograms are recorded as bipolar pairs. The reference electrodes are amplifier-dependent but are usually placed over bony scalp areas near the FCz or FC3 electrode positions.

A five-minute resting qEEG with eyes closed (EC) and a five-minute resting EEG with eyes open (EO) is performed with the participant seated comfortably in a sound-attenuated room. The participant views a fixation cross on a video monitor during the eyes-open periods and is instructed not to move their eyes or blink excessively. The resting qEEG is evaluated by means of spectral and coherence analysis, including spectral amplitudes and coherences in clinical frequency bands (delta, theta, alpha1, alpha2, beta1, beta2, beta2, beta3, high beta, gamma) and derived frequency measures (theta-beta ratio [TBR], beta-alpha ratio [BAR], alpha-slow wave index [ASI], dominant frequency, 1/f slope). qEEG is measured within 45 minutes of the planned dose time and again, 1, 2, 4, and 8-hours post-morning dose.

### Example 3: CYP and P-Glycoprotein Interaction Potential

### Rationale

Complex polypharmacy is common in patients with developmental and epileptic encephalopathies (DEE), therefore, avoiding drug-drug interactions (DDI) is particularly important in this population. Many antiseizure medications (ASMs) are affected by CYP enzyme inhibitors, notably CYP2D6 (fenfluramine, carbamazepine), CYP3A4 (clobazam, cannabidiol, felbamate, carbamazepine), and CYP2C19 (fenfluramine, cannabidiol, phenobarbital, phenytoin). In addition, many patients use 3 or more ASMs concomitantly, some of which are CYP inhibitors themselves. P-glycoprotein (P-gp)-mediated efflux and renal transporters can also cause undesirable pharmacokinetic (PK) effects. As such, a favorable PK profile of Compound 1 involves avoidance of CYP metabolism, P-gp efflux, and renal transporters, and reliance instead on UDP-glucuronosyltransferase (UGT) disposition.

### Methods

The study was designed to: (a) confirm metabolism of Compound 1 via glucuronidation by UGT; (b) assess Compound 1 disposition and potential to be affected by renal transporters; and (c) characterize the likelihood of Compound 1 to be affected P-gp efflux or by DDIs through the CYP metabolic pathway. This open-label study of 19 healthy volunteers was conducted in 2 parts. In part 1, the UGT metabolic pathway and role of renal transporters was assessed using a single 12 mg dose of Compound 1 (administered as a liquid formulation; Table 2, above) in the presence of a UGT inhibitor (probenecid) and a renal transport inhibitor (cimetidine) compared with Compound 1 alone. In part 2, the PK of steady-state 12 mg TID Compound 1 (administered as a liquid formulation; Table 2, above) was assessed with a CYP and P-gp inhibitor (quinidine), compared to Compound 1 alone. Serial plasma samples were collected in both parts of the study. Safety parameters were monitored throughout.

### Results

Part 1: Cₘₐₓ and AUC values for Compound 1 were higher in the presence of probenecid/cimetidine, as compared to Compound 1 alone. The observed ˜80% increase in Compound 1 exposure is consistent with, and supportive of, in vitro data showing disposition of Compound 1 via UGT, and a low likelihood of being affected by renal transport inhibitors.

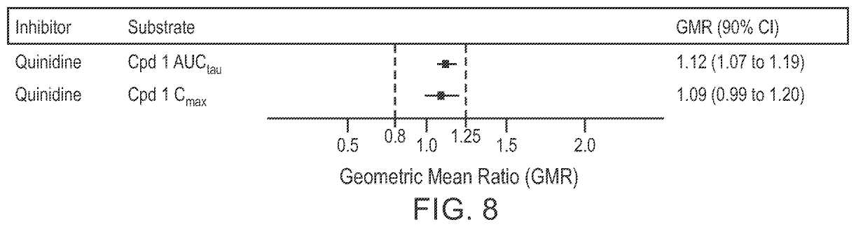

Part 2: Cₘₐₓ and AUC values for Compound 1 were 33.6 ng/mL and 167.4 h·ng/mL in the presence of quinidine, and 30.8 ng/mL and 148.9 h·ng/mL for Compound 1 alone. The lack of effect of quinidine on exposure indicates that Compound 1 is not a substrate for CYP3A4, CYP2D6, or P-gp. FIG. 8 shows the ratio of geometric least squares means comparing administration of Compound 1 alone to administration with quinidine. The potential for Compound 1 to be affected by inhibition of CYP219 and CYP2C9 was not assessed because in vitro data indicated that these are unlikely metabolic pathways for Compound 1. Compound 1 was safe and generally well-tolerated, alone or in combination with other probe substrates.

The results show that Compound 1 is unlikely to be subject to clinically significant CYP-mediated DDIs. Moreover, these results support a low likelihood of P-gp or renal transporter interactions in the disposition of Compound 1.

### Example 4: Anti-Seizure Activity in Preclinical Seizure Models

### Rationale

5-HT₂ receptor agonists have demonstrated efficacy for a variety of seizure types and seizure disorders. Compound 1 is a potent, selective 5-HT_{2C} superagonist. Proof of concept for the utility of Compound 1 across a range of seizure etiologies was established in zebrafish and mouse model systems. Due to rapid reproductive capacity and ease of genetic manipulation, both zebrafish and mice are highly useful model systems for studying many human diseases. Both have been validated as experimental models for seizures and epilepsy and show sensitivity to many classes of anti-seizure medications.

### Methods

For zebrafish studies, locomotor activity of individual larvae in 96-well plates was tracked with an automated tracking device. Local field potentials were recorded via non-invasive surface recordings from the skin above the optic tectum, and epileptiform activity was quantified.

Experiment 1: Mutations in the human SCN1A gene, which encodes for a voltage-gated sodium channel α-subunit, have been associated with the genetic epilepsy known as Dravet Syndrome. Zebrafish larvae containing mutations in the fish ortholog gene (scn1Lab^{-/-}) were treated with Compound 1 or vehicle, and motor behavior and brain epileptiform activity were measured.

Experiment 2: Wild-type zebrafish larvae were treated with ethyl ketopentenoate (EKP), which reduces the synthesis of the inhibitory neurotransmitter GABA, to induce generalized seizures. EKP-treated larvae were exposed to Compound 1, and brain epileptiform activity was recorded.

Experiment 3: Wild-type zebrafish larvae were treated with kainic acid (KA), a cyclic analog of L-glutamate that binds to and activates excitatory glutamate receptors, to induce acute and chronic seizures in zebrafish in a model of temporal lobe epilepsy. KA-treated larvae were exposed to Compound 1, and brain epileptiform activity was recorded.

Experiment 4: Mice were given intravenous pentylenetetrazol (PTZ), an antagonist of the GABA-A receptor, to produce myoclonic and tonic-clonic seizures in a model of generalized epilepsy. Compound 1 was administered orally prior to PTZ administration, and time to the first myoclonic twitch or onset of generalized clonus was recorded.

### Results

Experiment 1: Compound 1 treatment reduced locomotor activity and both the frequency and the mean cumulative duration of epileptiform events (84% and 85%, respectively).

Experiment 2: Compound 1 treatment reduced brain seizure activity an average of 69.1%.

Experiment 3: Compound 1 treatment produced an 82.4% reduction in brain seizure activity.

Experiment 4: Compound 1 administration produced a dose-dependent increase in the time to the first myoclonic twitch and the time of onset to generalized clonus.

The results show that Compound 1 broadly reduces a wide variety of seizure activities stemming from numerous underlying causes including genetic mutations in neuronal sodium channels, reduced GABAergic signaling, and excessive glutamatergic excitation. These data support the usefulness of Compound 1 in treatment of heterogeneous seizure disorders, for example, in DEE patients with heterogeneous underlying pathologies.

### Example 5: Multiple-Dose Study in Healthy Adult Subjects

The Phase 1, open-label, multiple-dose study of Example 1 was conducted for Cohorts 1-3, 4A, and 4B.

48 healthy subjects ages 18 to 55 years, inclusive, and having body mass index of 18.5-30.0 kg/m² and a weight of at least 50 kg were enrolled. Subjects were enrolled to 1 of 5 dosage regimens. All dosage regimens included up-titration and taper-down phases. Compound 1 was administered according to the dosage regimens of Table 6:

TABLE 6
Schedule of Dosing

| Cohort | Dose # | D1 | D2 | D3 | D4 | D5 | D6 | D7 | D8 |
|---|---|---|---|---|---|---|---|---|---|
| 1 | 1 | 3 | 3 | 6 | 6 | 6 | 6 | 6 | 6 |
| (6 mg TID) | 2 | 3 | 3 | 6 | 6 | 6 | 6 | 6 | 6 |
| | 3 | 3 | 3 | 6 | 6 | 6 | 6 | 6 | 6 |
| 2 | 1 | 6 | 6 | 12 | 12 | 12 | 12 | 12 | 12 |
| (12 mg TID) | 2 | 6 | 6 | 12 | 12 | 12 | 12 | 12 | 12 |
| | 3 | 6 | 6 | 12 | 12 | 12 | 12 | 12 | 12 |
| 3 | 1 | 6 | 6 | 12 | 12 | 12 | 12 | 12 | 12 |
| (12 mg BID) | 2 | 6 | 6 | 12 | 12 | 12 | 12 | 12 | 12 |
| 4A | 1 | 6 | 6 | 12 | 12 | 15 | 15 | 15 | 15 |
| (15 mg BID)^{a} | 2 | 6 | 6 | 12 | 12 | 15 | 15 | 15 | 15 |
| 4B | 1 | 6 | 6 | 12 | 12 | 18 | 18 | 18 | 18 |
| (18 mg BID)^{a} | 2 | 6 | 6 | 12 | 12 | 18 | 18 | 18 | 18 |

| Cohort | Dose # | D9 | D10 | D11 | D12 | D13 | D14 | D15 | D16 |
|---|---|---|---|---|---|---|---|---|---|
| 1 | 1 | 6 | 6 | 6 | 6 | 3 | 3 | 3 | 3 |
| (6 mg | 2 | 6 | 6 | — | — | — | — | — | — |
| TID) | 3 | 6 | 6 | 6 | 6 | 3 | 3 | — | — |
| 2 | 1 | 12 | 12 | 12 | 12 | 6 | 6 | 3 | 3 |
| (12 mg | 2 | 12 | 12 | — | — | — | — | — | — |
| TID) | 3 | 12 | 12 | 12 | 6 | 6 | 3 | — | — |
| 3 | 1 | 12 | 12 | 12 | 6 | 3 | 3 | 3 | 3 |
| (12 mg | 2 | 12 | 12 | 6 | 6 | 3 | 3 | — | — |
| BID) | | | | | | | | | |
| 4A | 1 | 15 | 15 | 15 | 9 | 4.5 | 4.5 | 4.5 | 4.5 |
| (15 mg | 2 | 15 | 15 | 9 | 9 | 4.5 | 4.5 | — | — |
| BID)^{a} | | | | | | | | | |
| 4B | 1 | 18 | 18 | 18 | 9 | 4.5 | 4.5 | 4.5 | 4.5 |
| (18 mg | 2 | 18 | 18 | 9 | 9 | 4.5 | 4.5 | — | — |
| BID)^{a} | | | | | | | | | |

— = subjects were not dosed; D = day; AM = morning; BID = twice daily; TID = thrice daily.
Note:
For Cohorts 1 to 3: D1 to D2: up-titration (half of the target treatment dose); D3 to D11 AM dose (target treatment dose); taper (half of the previous dose) began on Day 11 after the AM dose.
Note:
For Cohorts 4A and 4B: D1 to D2: up-titration (6 mg); D3 to D4: up-titration (12 mg); D5 to D11 AM dose (target dose); taper began on D11 after the AM dose.
^{a}A minimum of 4 subjects (Cohort 4A) were up-titrated to 15 mg BID on D5 and completed the D11 AM dose before the remaining subjects (Cohort 4B) were up-titrated to the target dose of 18 mg BID.

Subjects received all Compound 1 doses as a liquid formulation (Table 2, above) for oral administration immediately followed by approximately 240 mL of water. Morning doses were administered following a standard light breakfast (e.g., milk and cereal, toast, and fruit) on all days. TID dosing (three times a day) was approximately 8 hours apart, and BID dosing (two times a day) was 12 hours apart. Subjects fasted from midnight onwards, until the morning breakfast each day. There was no fluid restriction during the study, but subjects were not to consume excessive amounts of fluid on any day.

For all cohorts, the study consisted of a Screening period (Day −28 to −2), an on-site period from Day −1 to Day 16 and a 10-day postdosing follow-up visit (Day 25). Subjects were admitted to the clinic on Day −1 of the assessment period and remained confined to the clinic until completion of the assessment period (or longer if the Investigator deemed it is clinically indicated for safety follow-up). Safety was assessed continuously from signing of informed consent form through follow up visit. Safety assessments including cardiac troponins, vital signs, 12-lead ECGs (electrocardiograms) were performed at screening and daily from Day −1 to Day 16, and follow-up. C-SSRS (Columbia-suicide severity rating scale) was assessed at screening, Days −1, 11, 16, and 25.

Study assessments related to PK and PD endpoints were as follows:
Serial CSF samples: Day 11
Serial sampling for plasma protein binding: Day 11
Serial qEEGs: Days −1, 1, 3 (cohorts 1-3), 5 (cohorts 4A, 4B), and 10
Trough qEEG: Day 16
Serial serum prolactin samples: Days 1 and 10
Serial plasma PK samples: Days 1, 3, 5 (cohorts 4A, 4B), 8-11
Trough plasma PK sample: Days 2, 4-7, and 12-16
Bladder ultrasounds were performed on Day −1, 2, 8, and 9
Sampling for pharmacogenomics: Day 1

### Pharmacokinetics

Pharmacokinetic parameters for Compound 1 and its metabolites were determined from the CSF and plasma concentration-time profiles for all evaluable subjects. The parameters are set forth in Table 7, below.

TABLE 7
Pharmacokinetic Parameter Definitions

| Parameter | Definition |
|---|---|
| Cₘₐₓ | Maximum observed concentration in a profile |
| Cₘₐₓ/ | Dose-normalized Cₘₐₓ |
| Dose | |
| C_{avg} | Average observed concentration within the dosing |
| | cycle after multiple dosing, defined as AUCₜₐᵤ/tau |
| C_{trough} | Pre-morning dose concentration |
| AUCₜₐᵤ | Area under the concentration-time curve over the |
| | dosing interval tau |
| AUCₜₐᵤ/ | Dose-normalized AUCₜₐᵤ |
| Dose | |
| AUC_{0-inf} | Area under the concentration-time curve extrapolated |
| | to infinity |
| CL/F | Apparent clearance, calculated as dose/AUC (AUC_{0-inf} |
| | on Day 1 and AUCₜₐᵤ on Days 8, 9, and 10) |
| Tₘₐₓ | Time to the 1^{st} occurrence of maximum observed |
| | concentration in a profile |
| λ_{z} | Apparent elimination rate constant, computed |
| | by the log-linear regression of the terminal log- |
| | linear segment of the plasma concentration-time curve |
| T_{1/2} (h) | Apparent terminal half-life, calculated as t_{1/2} = ln(2)/λ_{z} |
| MPR | Molecular weight-adjusted metabolite-to-parent ratio |
| AUCₜₐᵤ | of AUCₜₐᵤ |
| MPR | Molecular weight-adjusted metabolite-to-parent ratio |
| Cₘₐₓ | of Cₘₐₓ |

CSF to free drug plasma ratios (CSF/P) of Cₘₐₓ, C_{trough}, and AUCₜₐᵤ were also determined for Compound 1.

### Plasma PK of Compound 1

Plasma PK parameters were obtained for Compound 1 on the days noted above.

Compound 1 pre-dose concentrations in plasma were below the limit of quantification (BLQ) across all 5 cohorts on Day 1, with quantifiable Compound 1 concentrations observed one hour after dosing. On Day 1, after a single 3 mg dose of Compound 1 (Cohort 1), the geometric mean Cₘₐₓ of 4.7 ng/mL was reached at a median time of 1.00 hours, an apparent terminal half-life of 3.1 hours, and a geometric mean AUC_{0-inf} of 21.3 h*ng/mL. After a single 6 mg dose of Compound 1 (Cohorts 2, 3, 4A, and 4B) the geometric mean Cₘₐₓ ranged between 7.8 and 12.4 ng/mL, which was reached at a median time between 0.98 and 1.14 hours, with an apparent terminal half-life between 3.1 and 3.3 hours, and a geometric mean AUC_{0-inf} between 36.5 and 51.9 h*ng/mL across the 4 cohorts. Comparing Cohorts 2, 3, 4A, and 4B after a single 6 mg dose of Compound 1, Cohort 4A appeared to exhibit lower exposure PK parameters compared to the other 3 cohorts.

On Day 3, Cohort 1 was titrated up to 6 mg TID, Cohort 2 was titrated up to 12 mg BID, and Cohorts 3, 4A, and 4B were titrated up to 12 mg TID. On Day 5, Cohort 4A was titrated up to 15 mg TID, and Cohort 4B was titrated up to 18 mg TID. Due to the increases in dose, steady state had not yet been reached.

All cohorts reached steady-state exposure to Compound 1 by Day 8. A summary of Compound 1 plasma PK parameters on Days 8-10 is provided in Tables 8-10, respectively.

TABLE 8
Plasma PK Parameters, Day 8

| | Cohort | | | | |
|---|---|---|---|---|---|
| | 1 | 2 | 3 | 4A | 4B |
| | (N = 10) [n] | (N = 10) [n] | (N = 14) [n] | (N = 8) [n] | (N = 6) [n] |
| AUCₜₐᵤ | 77.07 (27.1) | 189.98 (45.1) | 156.21 (52.0) | 193.96 (27.9) | 230.36 (43.7) |
| (h*ng/mL) | [10] | [10] | [12] | [5] | [6] |
| AUC_{0-inf} | NC [0] | NC [0] | NC [0] | NC [0] | NC [0] |
| (h*ng/mL) | | | | | |
| C_{avg} (ng/mL) | 9.947 (2.6843) | 26.19 (14.697) | 14.42 (6.4875) | 16.64 (4.4047) | 20.62 (8.3690) |
| | [10] | [10] | [12] | [5] | [6] |
| Cₘₐₓ (ng/mL) | 16.55 (23.6) | 36.73 (44.8) | 30.29 (40.3) | 44.80 (36.1) | 43.22 (44.3) |
| | [10] | [10] | [12] | [5] | [6] |
| C_{trough} (ng/mL) | 5.150 (1.6616) | 14.96 (9.9859) | 4.892 (2.8780) | 5.557 (2.3832) | 7.427 (3.4830) |
| | [10] | [10] | [12] | [5] | [6] |
| CL/F (L/h) | 80.35 (21.282) | 67.87 (24.352) | 86.15 (46.458) | 79.73 (22.447) | 83.94 (34.338) |
| | [10] | [10] | [12] | [5] | [6] |
| Tₘₐₓ (h) | 1.54 (0.50, 2.52) | 1.01 (0.51, 2.50) | 1.01 (0.50, | 0.55 (0.50, | 0.99 (0.49, |
| | [10] | [10] | 2.23) [12] | 2.54) [5] | 2.00) [6] |
| T_{1/2} (h) | 4.490 (1.1046) | 4.743 (0.95528) | 4.321 | 5.364 (2.6770) | 5.514 (1.8774) |
| | [10] | [10] | (0.82910) [12] | [5] | [6] |
| AUCₜₐᵤ/Dose | 12.85 (27.1) | 15.83 (45.1) | 13.02 (52.0) | 12.93 (27.9) | 12.80 (43.7) |
| (h*ng/mL/mg) | [10] | [10] | [12] | [5] | [6] |
| Cₘₐₓ/Dose | 2.759 (23.6) | 3.061 (44.8) | 2.524 (40.3) | 2.987 (36.1) | 2.401 (44.3) |
| (ng/mL/mg) | [10] | [10] | [12] | [5] | [6] |

N = number of subjects in the Analysis Population;
n = number of subjects with non-missing PK data
Note:
AUCₜₐᵤ, AUC_{0-inf}, C_{avg}, Cₘₐₓ, and C_{trough} are presented as Geometric Mean (Geometric CV %);
Tₘₐₓ is presented as median (minimum, maximum); other parameters are presented as mean (SD).

TABLE 9
Plasma PK Parameters, Day 9

| | Cohort | | | | |
|---|---|---|---|---|---|
| | 1 | 2 | 3 | 4A | 4B |
| | (N = 10) [n] | (N = 10) [n] | (N = 14) [n] | (N = 8) [n] | (N = 6) [n] |
| AUCₜₐᵤ | 84.87 (29.5) | 193.55 (49.7) | 163.09 (48.4) | 207.60 (29.4) | 241.88 (52.0) |
| (h*ng/mL) | [10] | [10] | [12] | [5] | [6] |
| AUC_{0-inf} | NC [0] | NC [0] | NC [0] | NC [0] | NC [0] |
| (h*ng/mL) | | | | | |
| C_{avg} (ng/mL) | 11.03 (3.3822) | 27.22 (17.047) | 14.85 (6.1228) | 17.84 (4.6403) | 22.23 (10.604) |
| | [10] | [10] | [12] | [5] | [6] |
| Cₘₐₓ (ng/mL) | 18.42 (27.3) | 44.53 (42.4) | 37.18 (36.5) | 50.17 (32.6) | 47.71 (52.2) |
| | [10] | [10] | [12] | [5] | [6] |
| C_{trough} (ng/mL) | 5.181 (1.9717) | 14.74 (13.800) | 4.864 (2.7031) | 6.497 (2.3570) | 7.066 (4.3229) |
| | [10] | [10] | [12] | [5] | [6] |
| CL/F (L/h) | 73.30 (20.349) | 67.49 (26.346) | 81.56 (42.319) | 74.85 (23.600) | 81.96 (38.264) |
| | [10] | [10] | [12] | [5] | [6] |
| Tₘₐₓ (h) | 0.94 (0.51, 2.07) | 0.92 (0.52, 2.01) | 0.93 (0.49, | 0.57 (0.55, | 0.50 (0.49, |
| | [10] | [10] | 1.03) [12] | 0.94) [5] | 1.01) [6] |
| T_{1/2} (h) | 3.845 (0.84977) | 4.787 (2.7215) | 4.585 (0.97065) | 5.514 (1.9337) | 4.303 (1.5906) |
| | [10] | [10] | [12] | [5] | [6] |
| AUCₜₐᵤ/Dose | 14.15 (29.5) | 16.13 (49.7) | 13.59 (48.4) | 13.84 (29.4) | 13.44 (52.0) |
| (h*ng/mL/mg) | [10] | [10] | [12] | [5] | [6] |
| Cₘₐₓ/Dose | 3.070 (27.3) | 3.710 (42.4) | 3.098 (36.5) | 3.344 (32.6) | 2.650 (52.2) |
| (ng/mL/mg) | [10] | [10] | [12] | [5] | [6] |

N = number of subjects in the Analysis Population;
n = number of subjects with non-missing PK data
Note:
AUCₜₐᵤ, AUC_{0-inf}, C_{avg}, Cₘₐₓ, and C_{trough} are presented as Geometric Mean (Geometric CV %);
Tₘₐₓ is presented as median (minimum, maximum); other parameters are presented as mean (SD).

TABLE 10
Plasma PK Parameters, Day 10

| | Cohort | | | | |
|---|---|---|---|---|---|
| | 1 | 2 | 3 | 4A | 4B |
| | (N = 10) [n] | (N = 10) [n] | (N = 14) [n] | (N = 8) [n] | (N = 6) [n] |
| AUCₜₐᵤ | 82.61 (26.8) | 196.19 (48.1) | 153.82 (50.7) | 192.77 (34.1) | 224.21 (36.4) |
| (h*ng/mL) | [10] | [9] | [12] | [4] | [5] |
| AUC_{0-inf} | NC [0] | NC [0] | NC [0] | NC [0] | NC [0] |
| (h*ng/mL) | | | | | |
| C_{avg} (ng/mL) | 10.71 (2.8291) | 27.73 (18.586) | 14.14 (6.2142) | 16.70 (5.0410) | 19.64 (6.8876) |
| | [10] | [9] | [12] | [4] | [5] |
| Cₘₐₓ (ng/mL) | 16.46 (24.6) | 39.83 (51.1) | 31.33 (42.4) | 40.87 (34.0) | 53.02 (46.3) |
| | [10] | [9] | [12] | [5] | [6] |
| C_{trough} (ng/mL) | 5.730 (1.7982) | 14.30 (11.743) | 3.951 (2.4984) | 4.372 | 5.185 (2.4429) |
| | [10] | [9] | [12] | (0.70830) [4] | [5] |
| CL/F (L/h) | 77.30 (23.295) | 61.41 (19.610) | 86.88 (44.002) | 81.24 (28.741) | 84.30 (28.870) |
| | [7] | [7] | [12] | [4] | [5] |
| Tₘₐₓ (h) | 1.03 (0.97, 2.04) | 1.02 (0.96, 2.01) | 1.00 (0.93, | 1.06 (1.02, | 1.00 (0.99, |
| | [10] | [9] | 1.05) [12] | 2.00) [5] | 2.00) [6] |
| T_{1/2} (h) | 4.629 (0.66472) | 4.085 (0.78150) | 3.545 | 3.718 | 3.506 |
| | [7] | [7] | (0.71277) [12] | (0.76870) [4] | (0.43802) [5] |
| AUCₜₐᵤ/Dose | 13.77 (26.8) | 16.35 (48.1) | 12.82 (50.7) | 12.85 (34.1) | 12.46 (36.4) |
| (h*ng/mL/mg) | [10] | [9] | [12] | [4] | [5] |
| Cₘₐₓ/Dose | 2.744 (24.6) | 3.319 (51.1) | 2.611 (42.4) | 2.725 (34.0) | 2.945 (46.3) |
| (ng/mL/mg) | [10] | [9] | [12] | [5] | [6] |

N = number of subjects in the Analysis Population;
n = number of subjects with non-missing PK data
Note:
AUCₜₐᵤ, AUC_{0-inf}, C_{avg}, Cₘₐₓ, and C_{trough} are presented as Geometric Mean (Geometric CV %);
Tₘₐₓ is presented as median (minimum, maximum); other parameters are presented as mean (SD).

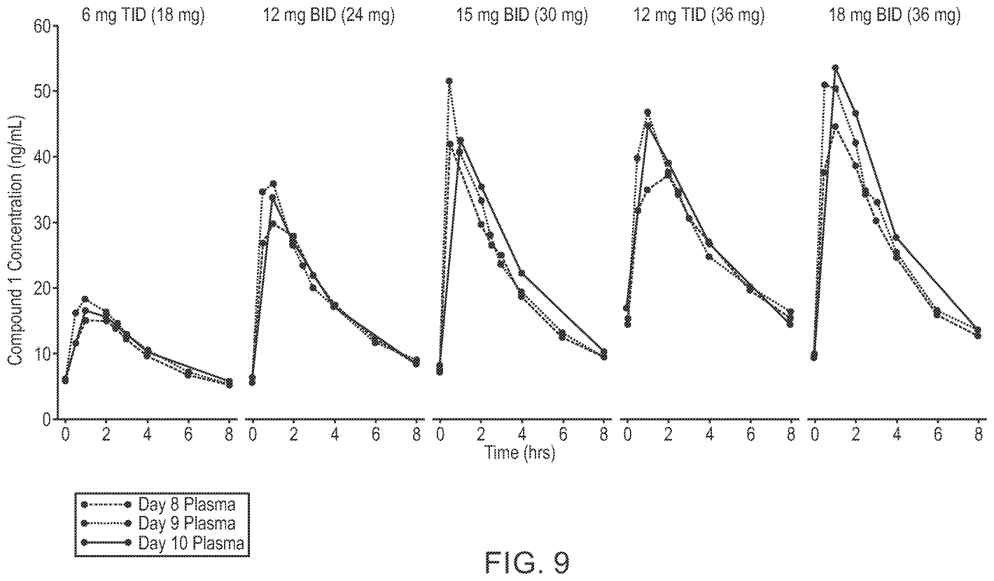

Mean concentration profiles of Compound 1 in plasma are shown in FIG. 9. The results demonstrated a linear increase in plasma concentration across daily doses, and consistent pharmacokinetics at steady state.

Exposure PK parameters appeared to be dose-proportional between 6 mg TID and 12 mg TID, as well as between 12 mg BID and 18 mg BID. At steady state, the half-life of Compound 1 was approximately 5 hours.

### Plasma PK of Compound 1 (Excluding Subject 208)

One subject (Subject 208) from Cohort 2 (12 mg TID) had an unexpected PK profile (high concentrations in plasma for all PK days and CSF). At steady state, following a visual inspection of the concentration-time curves, the plasma concentration for the 12 mg TID cohort, excluding Subject 208, showed a similar maximum concentration compared to the 12 mg BID cohort and similar concentrations to the 15 mg BID cohort in the terminal phase.

### Plasma PK of Compound 1 Metabolites

Plasma PK parameters were obtained for Compound 1 metabolites M9, M12, and M20 on the days noted above.

At steady state, M9 AUC and Cₘₐₓ increased less than proportionally between Cohorts 1 and 2. In the BID cohorts, AUC and Cₘₐₓ were higher in Cohort 3 compared to Cohort 4A, with a Tₘₐₓ of approximately 4 hours. MPR AUCₜₐᵤ and MPR Cₘₐₓ tended to be higher at lower doses within a given regimen, BID or TID.

At steady state, M12 AUC and Cₘₐₓ increased less than proportionally between Cohort 1 and Cohort 2. In the BID cohorts, AUC and Cₘₐₓ were higher in Cohort 3 compared to Cohort 4A, with a Tₘₐₓ of approximately 2 hours. MPR AUCₜₐᵤ and MPR Cₘₐₓ tended to be higher at lower doses within a given regimen, BID or TID.

At steady state, M20 AUC and Cₘₐₓ increased dose-proportionally between Cohort 1 and Cohort 2. In the BID cohorts, AUC and Cₘₐₓ also increased dose proportionally between Cohort 3 to Cohort 4B, with a Tₘₐₓ of approximately 1 hour. MPR AUCₜₐᵤ and MPR Cₘₐₓ were similar across dosing regimen. M20 levels were comparable between Cohorts 2 (12 mg TID) and 4B (18 mg BID).

### CSF PK of Compound 1

CSF PK parameters were obtained for Compound 1 on Day 11. A summary of CSF PK parameters for Compound 1 is presented in Table 11.

TABLE 11
CSF PK Parameters, Day 11

| | Cohort | | | | |
|---|---|---|---|---|---|
| | 1 | 2 | 3 | 4A | 4B |
| | (N = 10) [n] | (N = 10) [n] | (N = 14) [n] | (N = 8) [n] | (N = 6) [n] |
| AUCₜₐᵤ | 72.07 (25.7) | 167.4 (60.3) | 157.9 (34.0) | 171.2 (25.4) | 202.5 (30.1) |
| (h*ng/mL) | [9] | [7] | [9] | [5] | [5] |
| Cₘₐₓ | 11.88 (28.1) | 26.21 (57.4) | 21.34 (33.5) | 24.27 (32.3) | 30.05 (20.9) |
| (ng/mL) | [9] | [7] | [10] | [5] | [5] |
| C_{trough} | 6.524 (1.8619) | 18.69 (12.398) | 7.027 (3.2364) | 6.512 (1.3508) | 7.700 (3.5802) |
| (ng/mL) | [9] | [7] | [9] | [5] | [5] |
| Tₘₐₓ (h) | 2.52 (2.02, 6.07) | 3.04 (2.02, 4.01) | 2.53 (1.29, | 3.02 (2.07, | 3.06 (2.54, |
| | [9] | [7] | 6.14) [10] | 3.04) [5] | 4.03) [5] |
| C_{avg} | 9.261 (2.2660) | 24.61 (18.535) | 13.76 (4.0021) | 14.63 (3.7448) | 17.51 (5.7327) |
| (ng/mL) | [9] | [7] | [9] | [5] | [5] |
| CSF/P | 0.9422 (0.12350) | 0.8487 (0.083850) | 1.035 | 0.8899 | 0.9736 |
| AUCₜₐᵤ | [8] | [7] | (0.15691) [9] | (0.17018) [4] | (0.12068) [5] |
| CSF/P | 0.7640 (0.13958) | 0.6630 (0.087543) | 0.7408 | 0.6751 | 0.6946 |
| Cₘₐₓ | [8] | [7] | (0.17757) [10] | (0.20476) [5] | (0.19475) [5] |
| CSF/P | 1.263 (0.19312) | 1.312 (0.17194) | 1.917 | 1.482 | 1.694 |
| C_{trough} | [8] | [7] | (0.83340) [9] | (0.13895) [4] | (0.54202) [5] |

N = number of subjects in the Analysis Population;
n = number of subjects with non-missing PK data
Note:
AUCₜₐᵤ, AUC_{0-inf}, C_{avg}, Cₘₐₓ, and C_{trough} are presented as Geometric Mean (Geometric CV %);
Tₘₐₓ is presented as median (minimum, maximum); other parameters are presented as mean (SD).

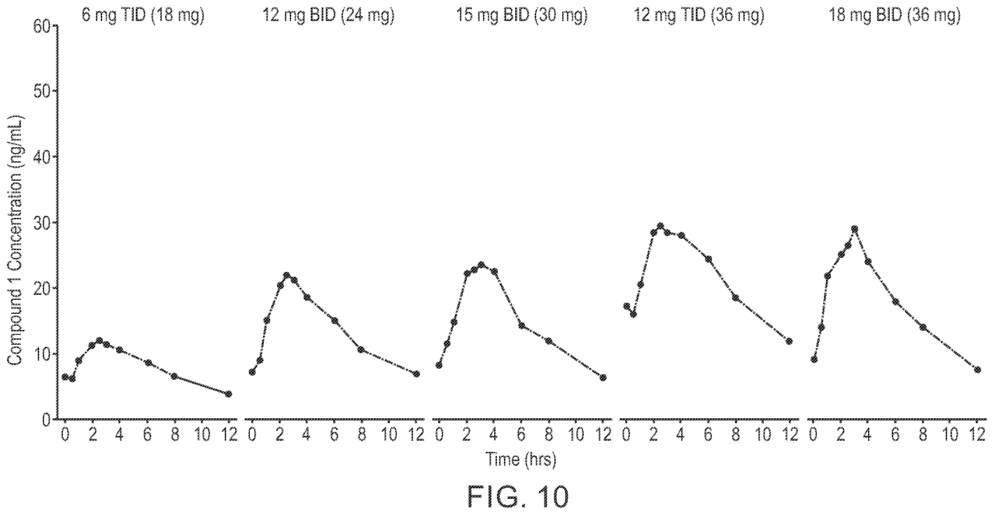

Mean concentration profiles of Compound 1 in CSF are shown in FIG. 10. The results demonstrated a linear increase in plasma concentration across daily doses, and consistent pharmacokinetics at steady state.

Compared to plasma PK parameters of Compound 1 on Day 10, a lower CSF Cₘₐₓ was reached at a later time, with a CSF Tₘₐₓ ranging from 2.52 hours to 3.06 hours. Compound 1 CSF Cₘₐₓ was lower than plasma Cₘₐₓ, with a CSF/P Cₘₐₓ ratio between 0.6630 and 0.7640 across all cohorts. Compound 1 CSF C_{trough} was higher than plasma C_{trough}, with a CSF/P C_{trough} ratio between 1.263 and 1.917 across all cohorts, suggesting a slower elimination half-life in the CSF compared to plasma.

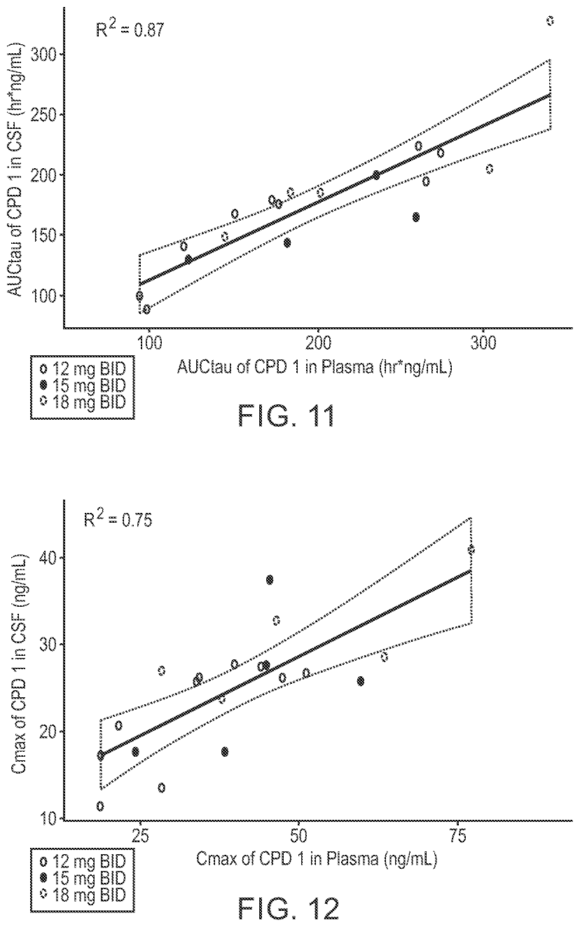

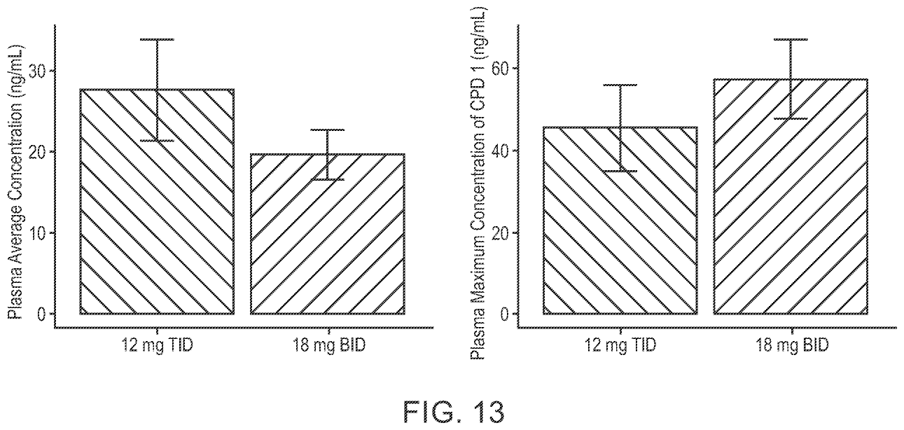

Across Cohorts 3, 4A, and 4B (BID dosing regimens), the observed PK in both plasma and CSF was dose linear, and CSF/P AUCₜₐᵤ was generally >0.84. There was a strong correlation of plasma and CSF PK parameters, including maximum concentration (Cₘₐₓ) and AUCₜₐᵤ (FIGS. 11 and 12). CSF/P Cₘₐₓ was lower for Cohorts 3, 4A, and 4B (BID dosing regimens) compared to Cohorts 1 and 2 (TID dosing regimens). Average plasma concentrations over the dosing interval were comparable (˜28%) between Cohort 2 (12 mg TID) and Cohort 4B (18 mg BID), as shown in FIG. 13.

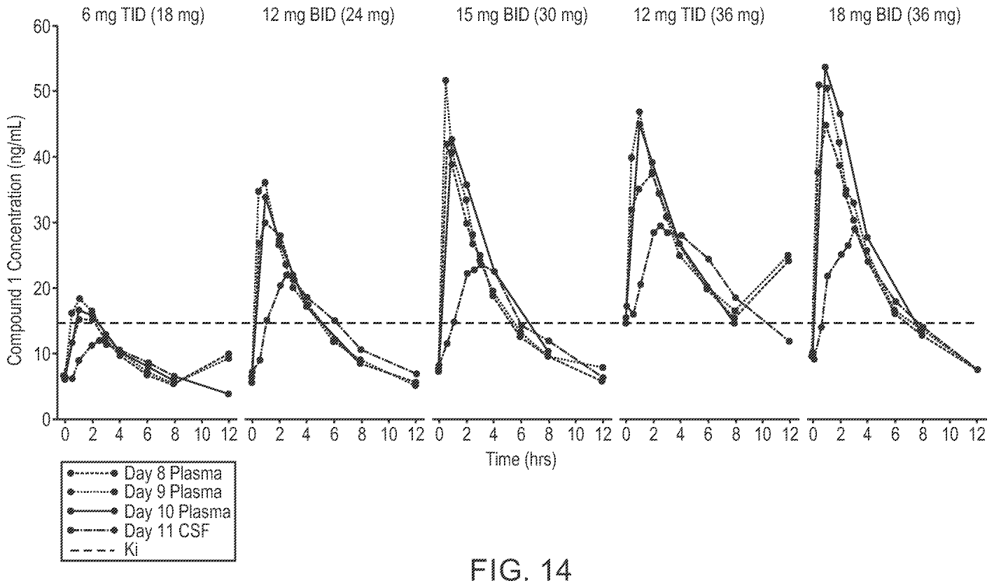

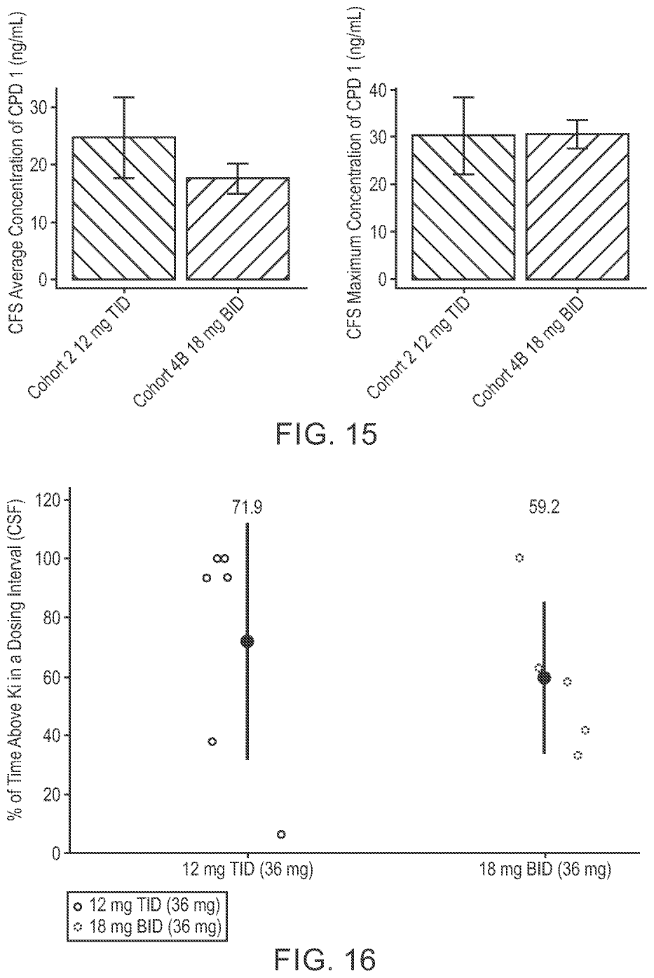

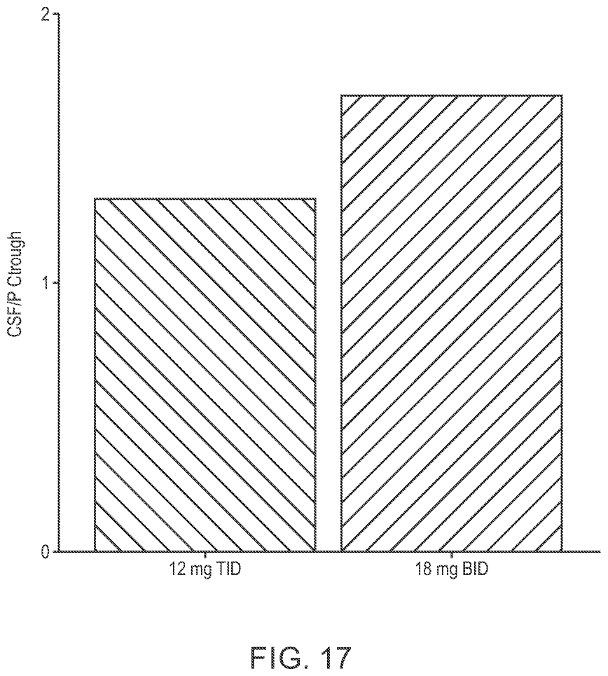

Mean concentration profiles of Compound 1 in CSF and plasma are compared with the relevant Kᵢ value (˜14 ng/mL) for 5-HT_{2C} agonism in FIG. 14. The results demonstrated a linear increase in plasma concentration across daily doses, and consistent pharmacokinetics at steady state. In CSF, Compound 1 concentrations generally exceeded the relevant Kᵢ value for 5-HT_{2C} agonism by ˜2-fold in Cohort 2 (12 mg TID) and Cohort 4B (18 mg BID) (FIG. 15). As shown in FIG. 16, percentage of time above Kᵢ for Cohort 2 and Cohort 4B was comparable, with Cohort 2 showing a larger spread and higher variability (Subject 208 was excluded).

Additionally, CSF/P C_{trough} was generally higher for BID regimens compared to TID. For example, FIG. 17 shows that the CSF/P C_{trough} for Cohort 4B (18 mg BID) is greater than that of Cohort 3 (12 mg TID), despite both cohorts receiving a total daily dosage of 36 mg of Compound 1. This higher ratio suggests that, for the same total daily dosage of Compound 1, BID administration can result in increased levels of Compound 1 in CSF- and in turn, increased exposure of Compound 1 to 5-HT_{2C} receptors, which are expressed in the brain.

### Plasma Protein Binding

Compound 1 plasma protein binding was assessed at 3 different timepoints on Day 11. For each individual timepoint, the average of the 3 values was used to derive the average free drug concentration, to compare or correlate with CSF concentration. As shown in Table 12, the mean unbound Compound 1 plasma fraction ranged from 91.3% to 96.9% across all cohorts.

TABLE 12
Compound 1 Plasma Protein Binding

| | Cohort | | | | |
|---|---|---|---|---|---|
| | 1 | 2 | 3 | 4A | 4B |
| | (N = 10) | (N = 10) | (N = 14) | (N = 8) | (N = 6) |
| Unbound | 0.969 | 0.968 | 0.930 | 0.913 | 0.926 |
| Fraction | (0.0392) | (0.0468) | (0.0503) | (0.0551) | (0.0464) |

### Pharmacodynamics

### Serum Prolactin

The PD parameters listed in Table 13 were derived for serum prolactin based on actual times and were calculated using noncompartmental analysis (NCA) methods, as data permitted.

TABLE 13
Pharmacodynamic Parameter Definitions

| Parameter | Definition |
|---|---|
| AUEC | Area under the serum concentration-time curve |
| | from time 0 to 8 hours, calculated by the linear |
| | trapezoidal method (Days 1 and 10) |
| Eₘₐₓ | Maximum observed serum concentration on |
| | Days 1 and 10 |
| Tₘₐₓ | Time of maximum observed serum concentration |
| | after multiple dosing. If the same maximum |
| | value occurred at more than 1 timepoint, Tₘₐₓ |
| | was defined as the 1st timepoint with this value |

Prolactin serum PD parameters on Days 1 and 10 are summarized in Tables 14 and 15, respectively.

TABLE 14
Prolactin Serum PK Parameters, Day 1

| | Cohort | | | | | | | | | |
|---|---|---|---|---|---|---|---|---|---|---|
| | 1 N = 10 | | 2 N = 10 | | 3 N = 14 | | 4A N = 8 | | 4B N = 6 | |
| | | Change | | Change | | Change | | Change | | Change |
| | | From | | From | | From | | From | | From |
| | Raw | Baseline | Raw | Baseline | Raw | Baseline | Raw | Baseline | Raw | Baseline |
| AUEC | 95.0 | 13.2 | 82.5 | 16.3 | 93.6 | 15.0 | 99.6 | 12.7 | 80.4 | NC |
| (h*ng/mL) | (39.8) | (150.4) | (33.6) | (77.1) | (42.2) | (212.5) | (51.7) | (357.1) | (14.2) | |
| Eₘₐₓ | 17.8 | 6.6 | 13.9 | 4.4 | 16.4 | 5.8 | 21.5 | 9.1 | 15.6 | 4.2 |
| (ng/mL) | (43.3) | (117.4) | (13.8) | (74.7) | (53.5) | (113.3) | (65.5) | (153.4) | (22.8) | (147.7) |
| Tₘₐₓ (h) | 7.84 | NC | 2.01 | NC | 1.56 | NC | 2.01 | NC | 0.48 | NC |
| | (0.00, | | (0.00, | | (0.00, | | (0.00, | | (0.00, | |
| | 7.89) | | 7.93) | | 7.86) | | 7.90) | | 7.84) | |

N = number of subjects in the Analysis Population
Note:
AUEC and Eₘₐₓ are presented as geometric mean (geometric CV %). Tₘₐₓ is presented as median (min, max).

TABLE 15
Prolactin Serum PK Parameters, Day 10

| | Cohort | | | | | | | | | |
|---|---|---|---|---|---|---|---|---|---|---|
| | 1 N = 10 | | 2 N = 10 | | 3 N = 14 | | 4A N = 8 | | 4B N = 6 | |
| | | Change | | Change | | Change | | Change | | Change |
| | | From | | From | | From | | From | | From |
| | Raw | Baseline | Raw | Baseline | Raw | Baseline | Raw | Baseline | Raw | Baseline |
| AUEC | 92.4 | 20.7 | 92.0 | 14.4 | 99.9 | 13.5 | 84.3 | NC | 99.4 | 17.8 |
| (h*ng/mL) | (42.8) | (80.6) | (27.1) | (410.4) | (38.6) | (252.3) | (22.6) | | (31.9) | (200.2) |
| Eₘₐₓ | 16.2 | 6.0 | 16.0 | 5.8 | 18.2 | 7.2 | 14.7 | 4.7 | 16.7 | 8.1 |
| (ng/mL) | (47.2) | (98.8) | (27.6) | (67.6) | (44.4) | (90.6) | (24.7) | (75.2) | (34.1) | (49.6) |
| Tₘₐₓ (h) | 3.00 | NC | 7.84 | NC | 1.01 | NC | 2.00 | NC | 1.10 | NC |
| | (0.00, | | (1.01, | | (0.00, | | (1.02, | | (0.00, | |
| | 7.90) | | 7.94) | | 7.86) | | 3.99) | | 7.84) | |

N = number of subjects in the Analysis Population
Note:
AUEC and Eₘₐₓ are presented as geometric mean (geometric CV %). Tₘₐₓ is presented as median (min, max).

Similar Eₘₐₓ values were observed across all cohorts, ranging between 14.7 to 18.2 ng/mL on Day 10. In general, higher Compound 1 exposure (Cₘₐₓ and AUCₜₐᵤ) was associated with greater change from baseline in prolactin AUEC and Eₘₐₓ for BID-dosed Cohorts 3, 4A, and 4B, whereas TID-dosed Cohorts 1 and 2 showed have an opposite correlation.

### EEG

After eligibility assessment, a screening EEG was performed, as part of the screening assessment to exclude subjects with clinically significant abnormalities. Screening EEGs were 20 minutes in duration and included hyperventilation and photic stimulation. They were scored as either within normal limits (0), or as abnormal and exclusionary (1).

All qEEG assessments were performed by registered EEG technicians using individually applied gold cup electrodes and state-of-the-art EEG recording systems. All tests were performed with the participant seated comfortably in a sound-attenuated room. In addition to the standard 19 EEG leads of the International 10/20 system, the left earlobe and right ear lobe were recorded as active leads, and the vertical and horizontal electro-oculograms were recorded as bipolar pairs. The reference electrodes were amplifier-dependent but were usually placed over bony scalp areas near the FCz or FC3 electrode positions.

A five-minute resting qEEG with eyes closed (EC) and a five-minute resting EEG with eyes open (EO) was performed with the participant seated comfortably in a sound-attenuated room. The participant viewed a fixation cross on a video monitor during the eyes-open periods and was instructed not to move their eyes or blink excessively. The resting qEEG was evaluated by means of spectral and coherence analysis, including spectral amplitudes and coherences in clinical frequency bands (delta, theta, alpha1, alpha2, beta1, beta2, beta2, beta3, high beta, gamma) and derived frequency measures (theta-beta ratio (TBR), beta-alpha ratio (BAR), alpha-slow wave index (ASI), dominant frequency, 1/f slope). Serial qEEGs were measured within 45 minutes of the planned dose time and again, 1, 2, 4, and 8-hours post-morning dose.

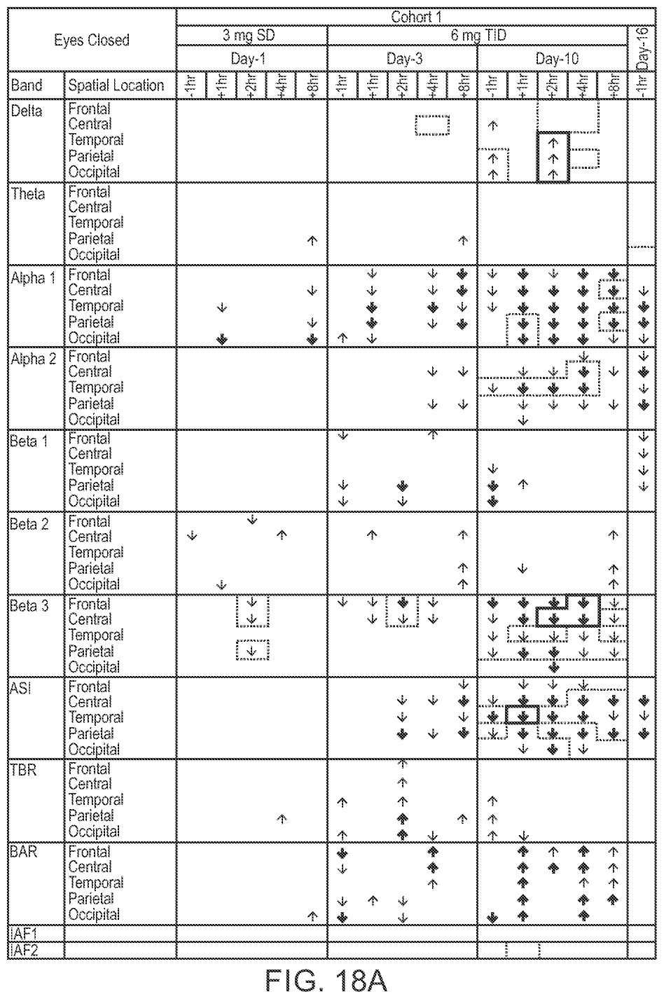

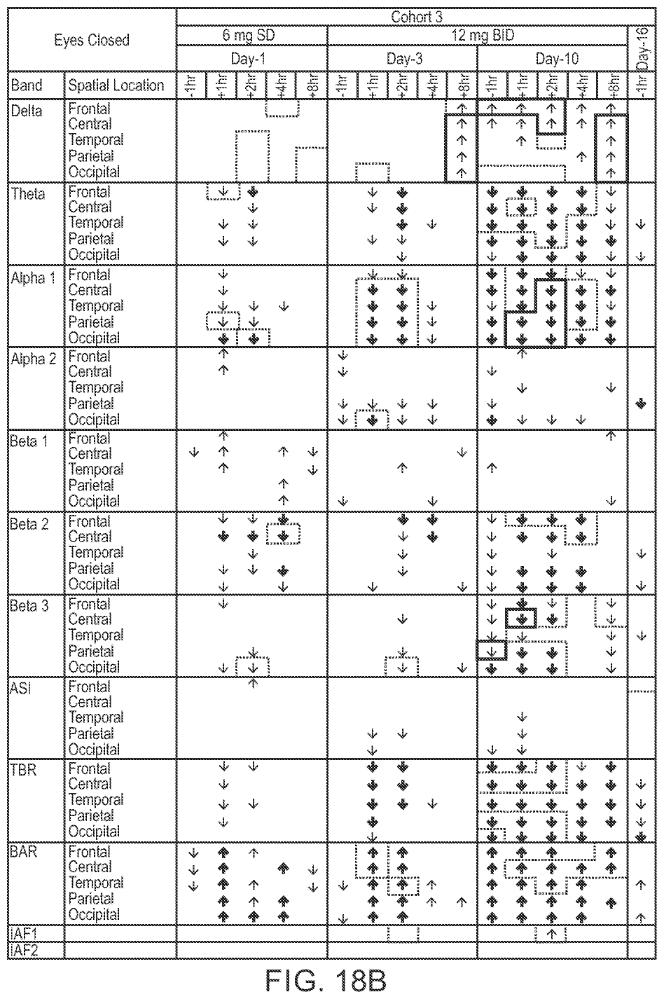

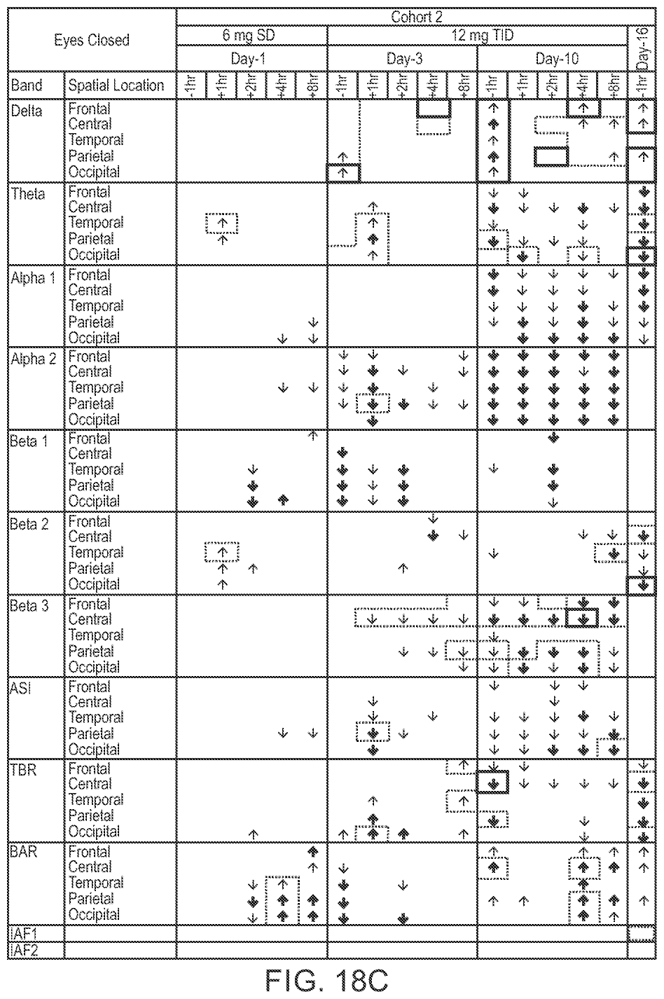

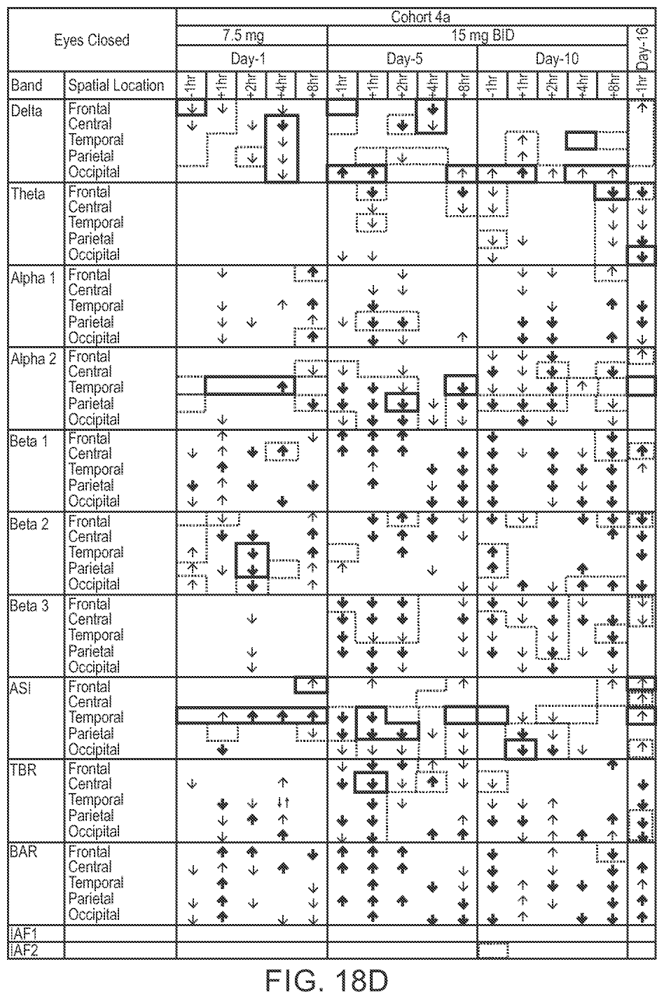

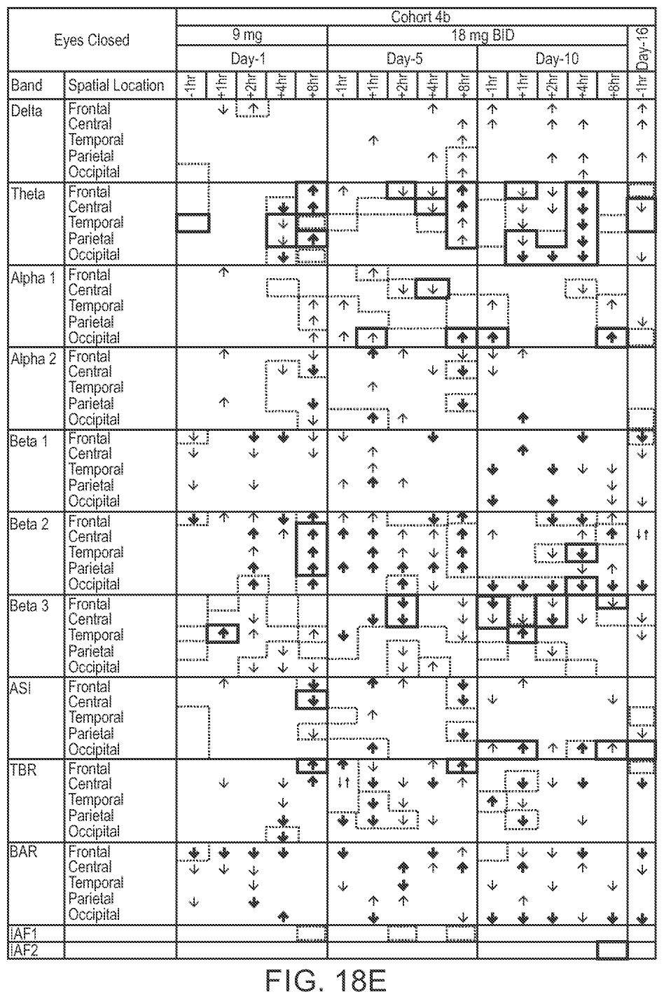

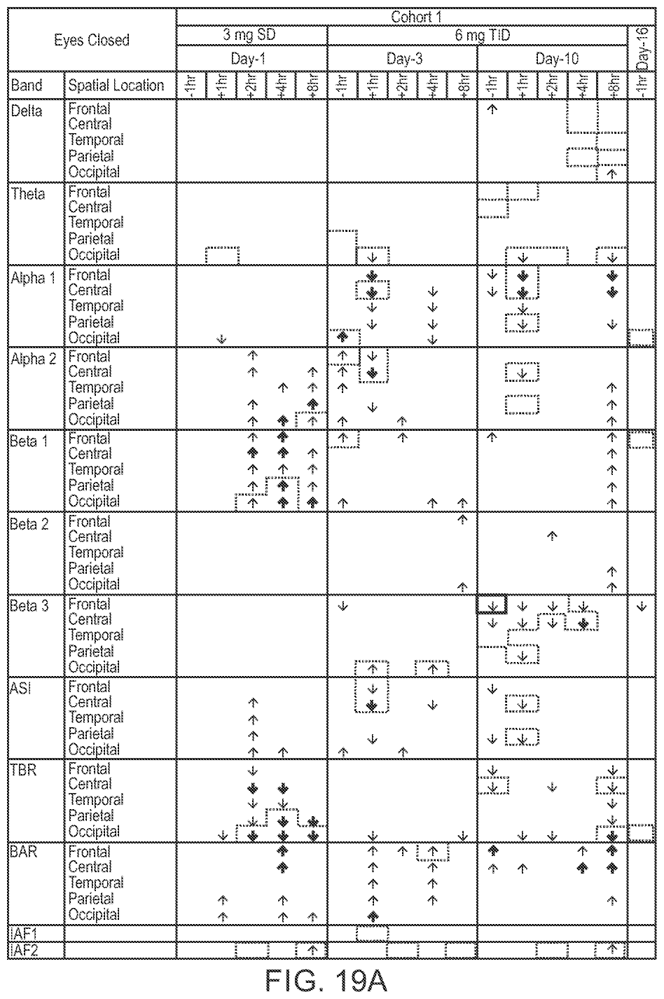

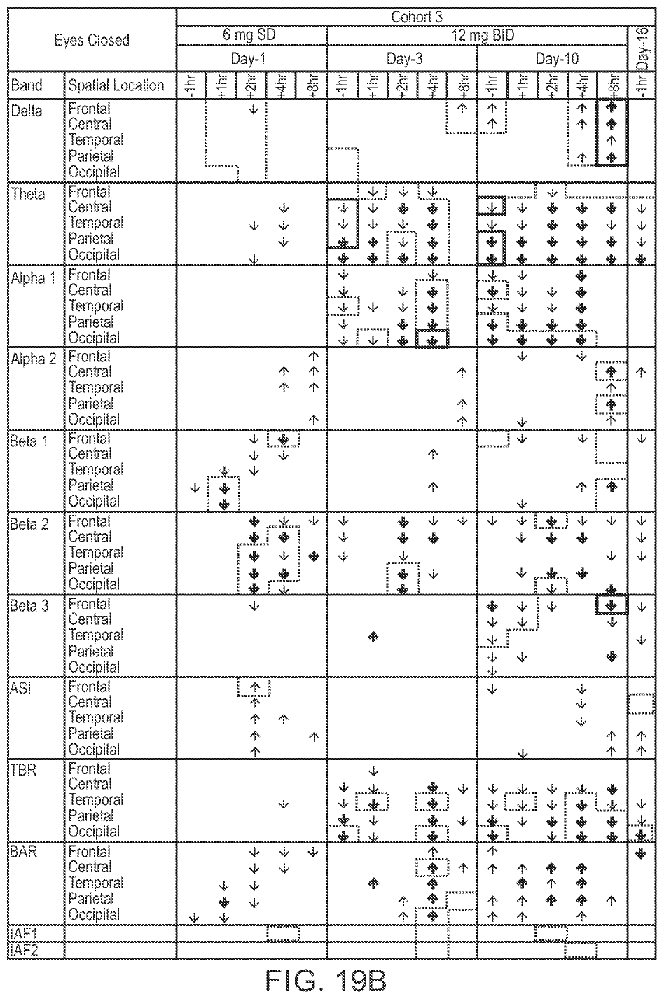

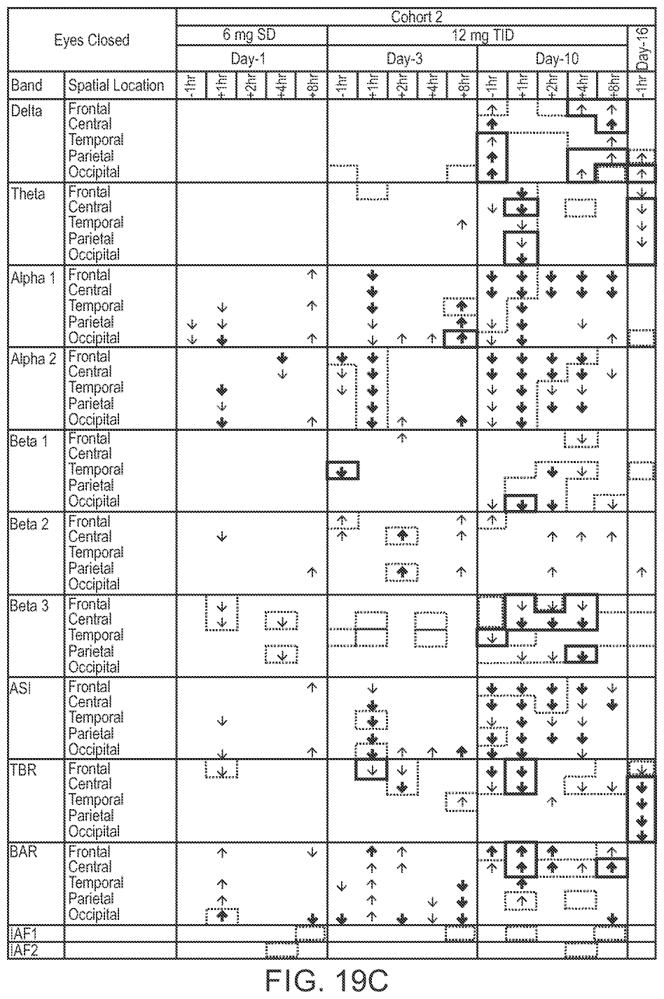

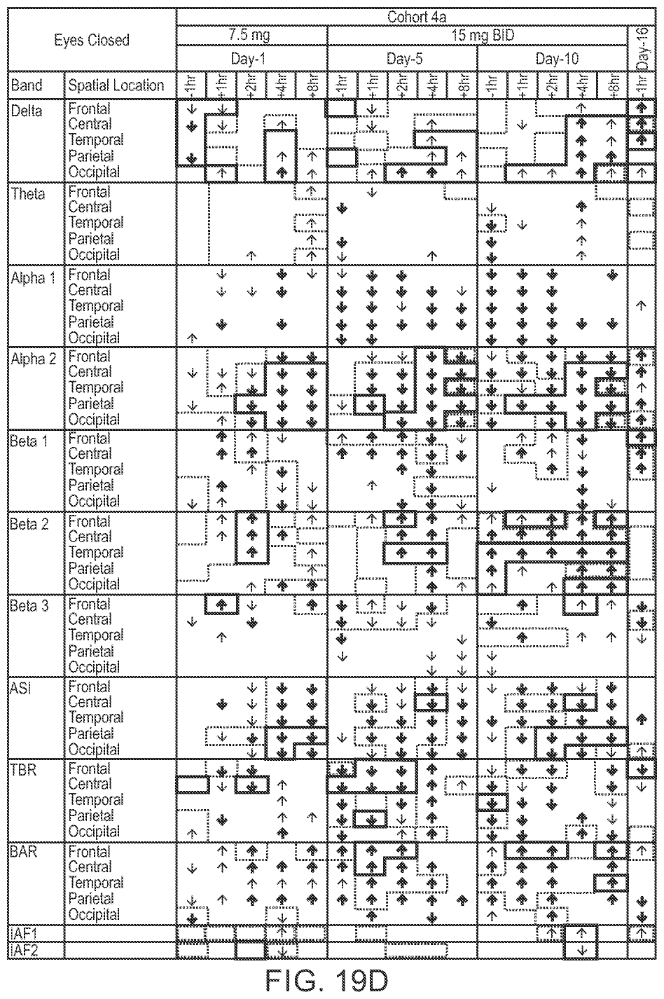

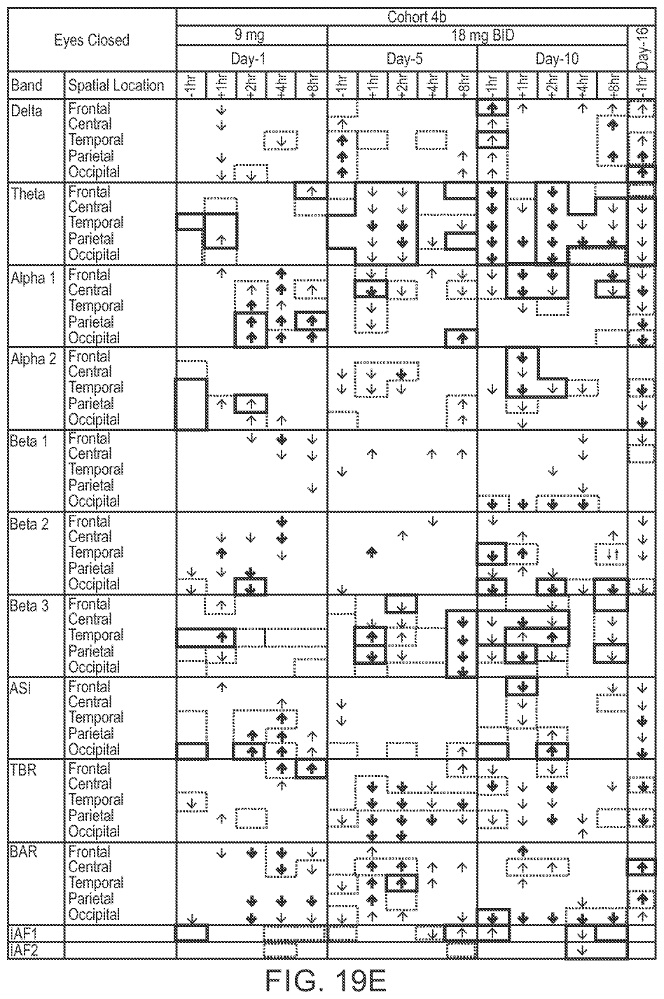

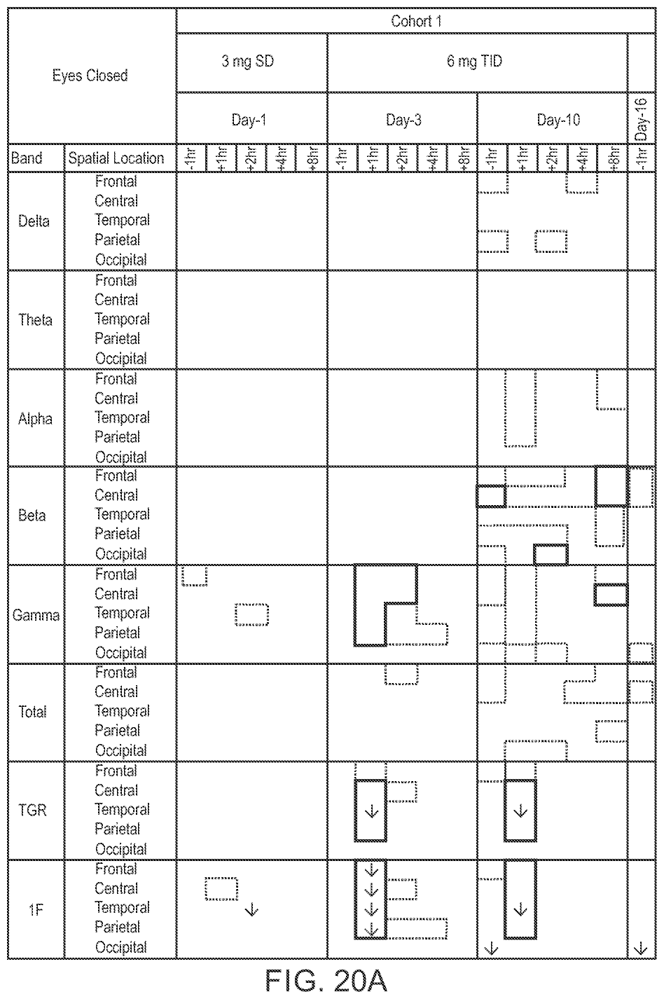

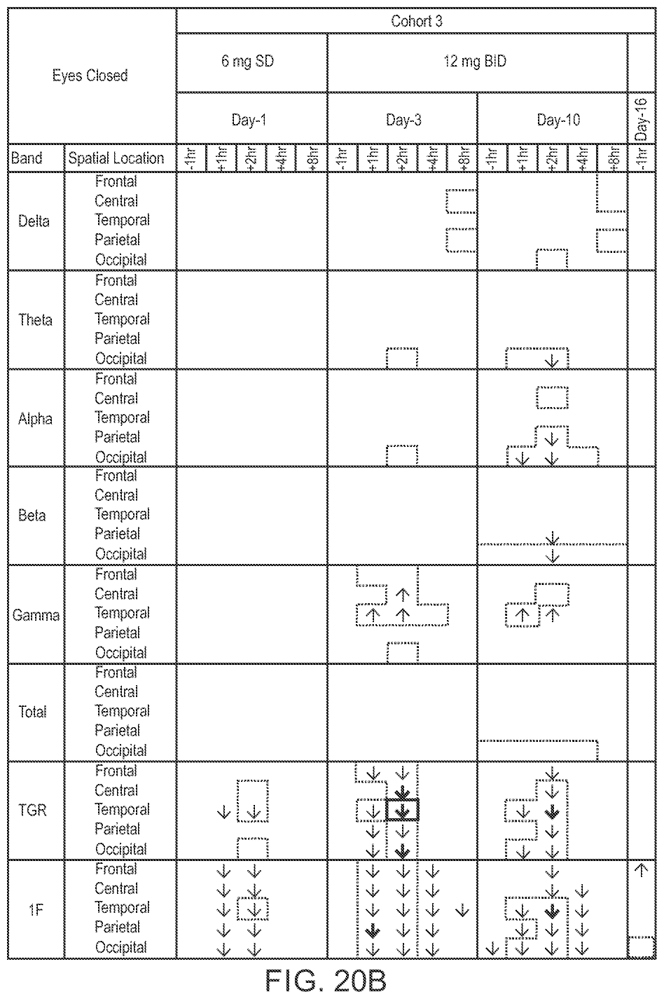

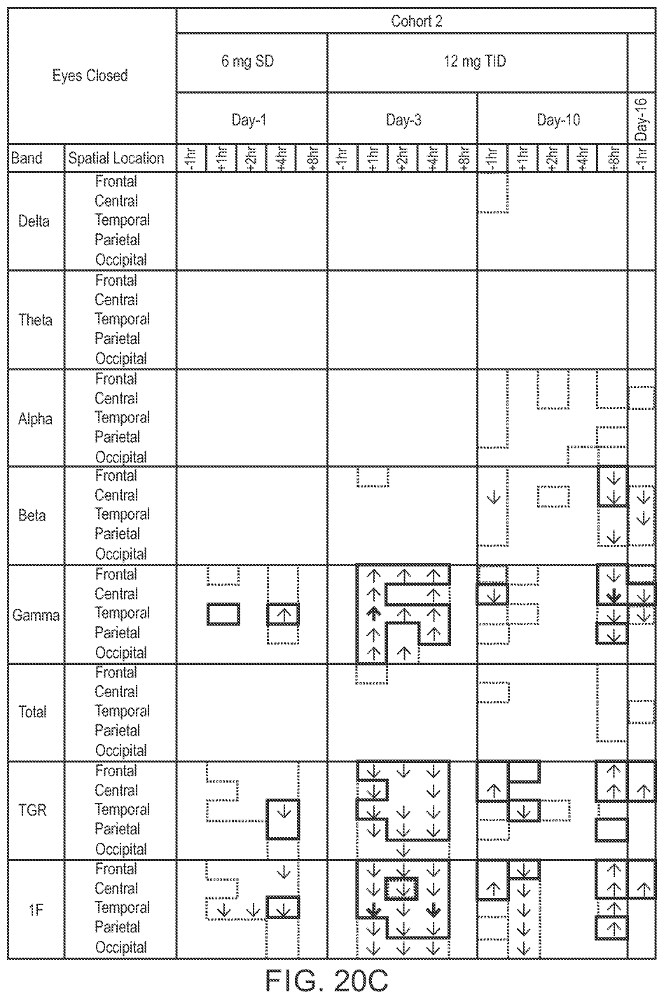

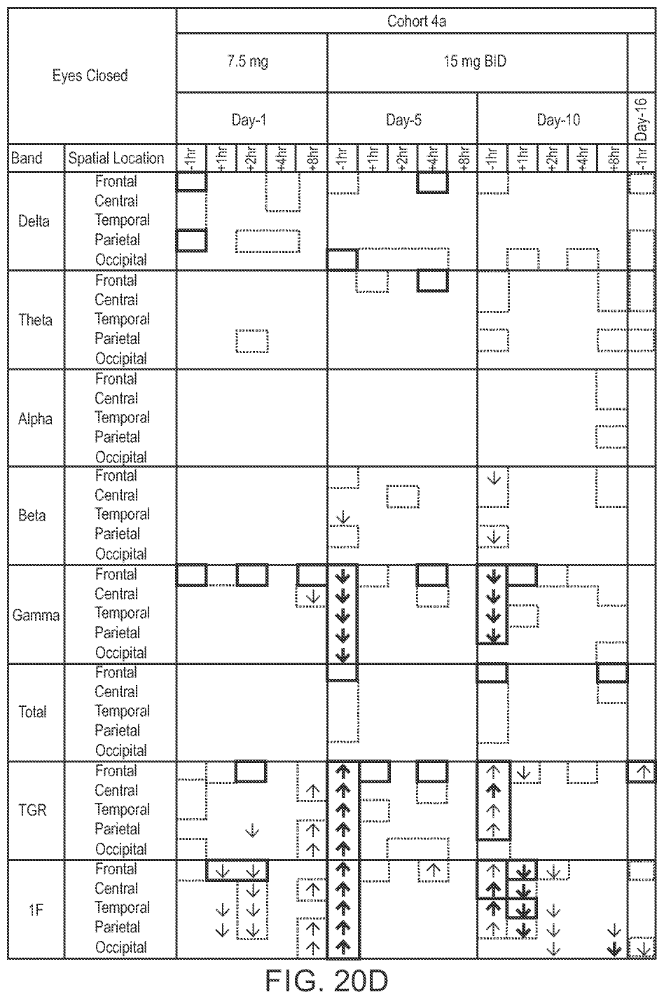

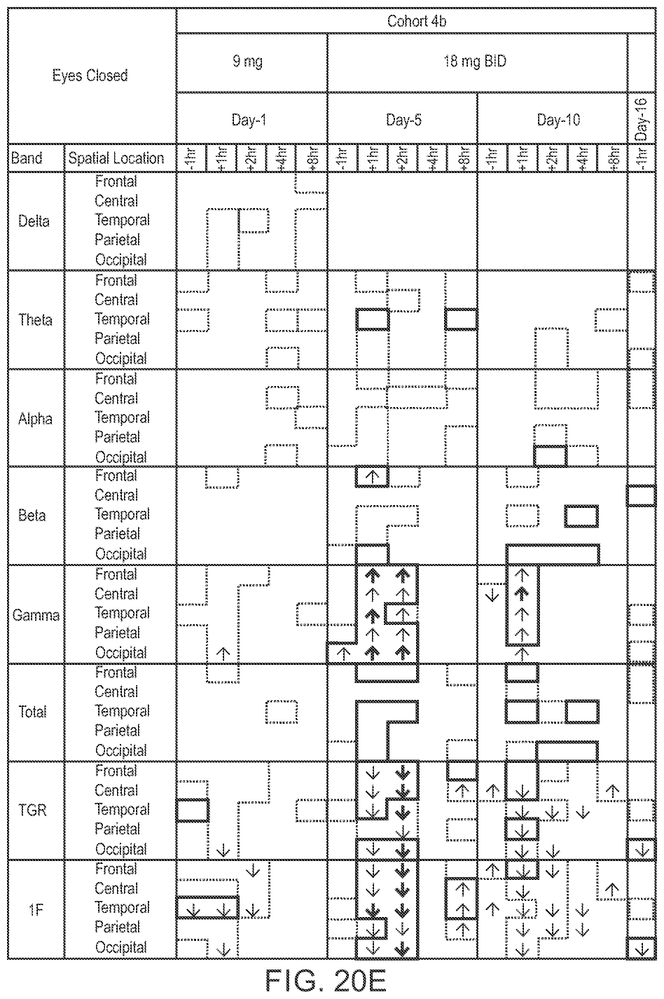

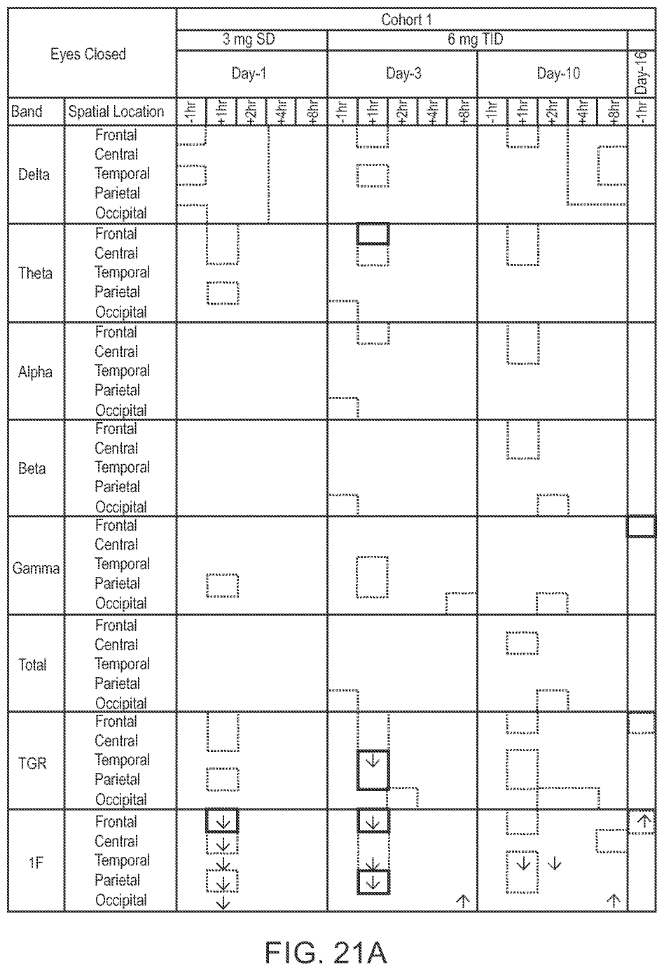

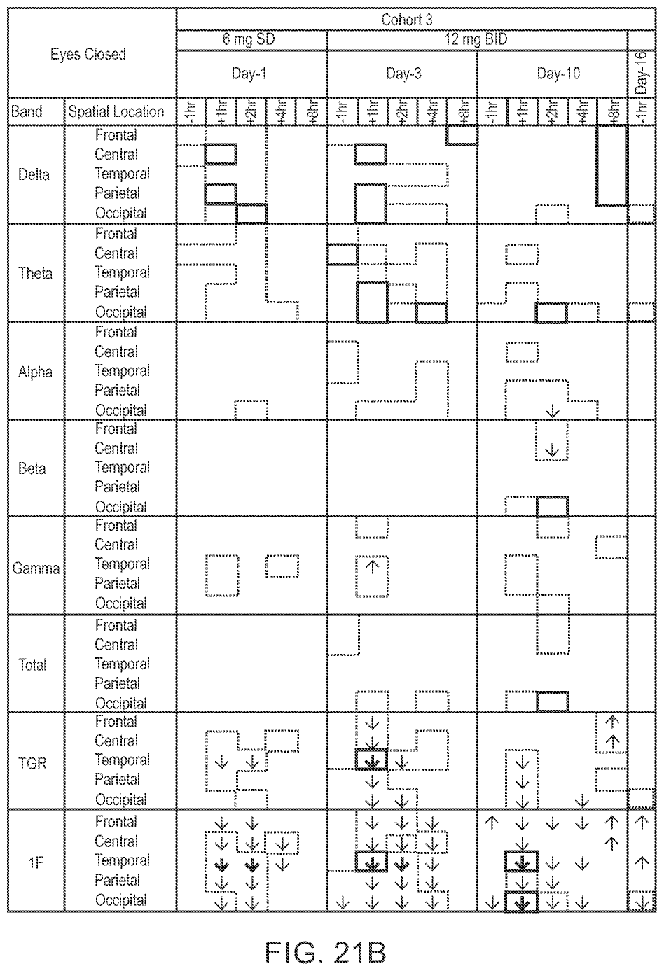

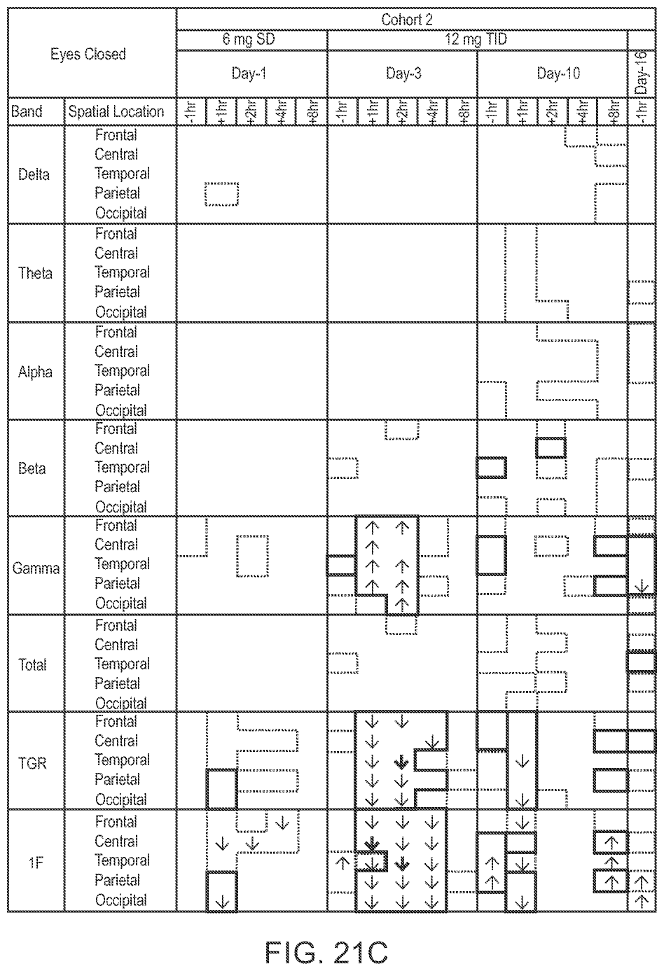

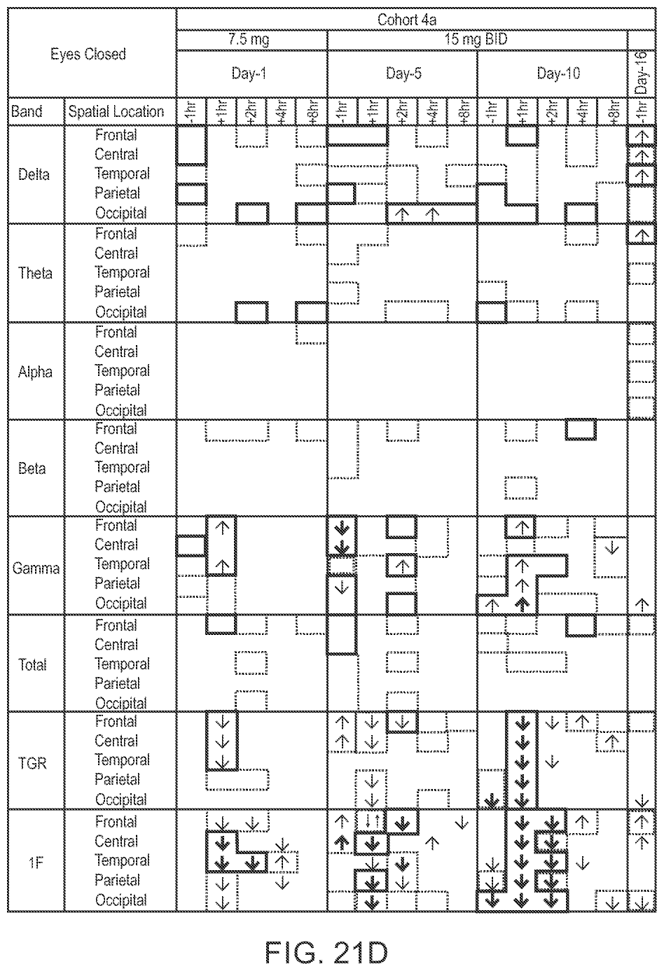

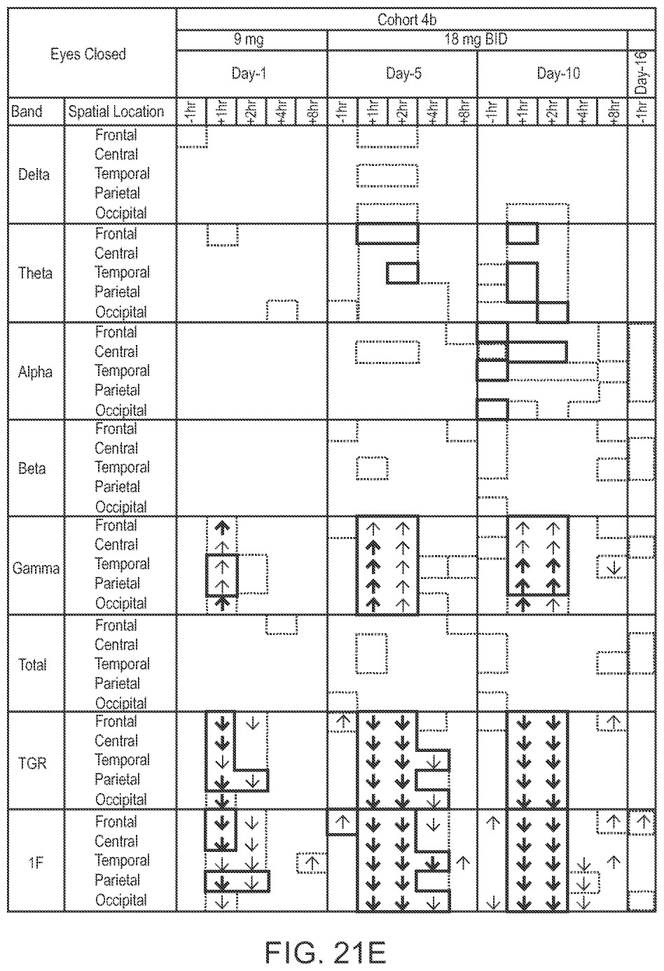

Representative qEEG results for all cohorts are shown in FIGS. 18-21. FIGS. 18 and 19 show changes in EC and EO oscillatory band parameters detected by qEEG, respectively. FIGS. 20 and 21 show changes in EC and EO fractal band parameters detected by qEEG, respectively. Small and large salient contrasts (≥10%, ≥15%) are indicated by light and bold arrows (down=decrease, up=increase) respectively. Small and large salient Cohen's d values (≥0.5, ≥0.8) are indicated by dashed and solid boxes, respectively. Days and timepoints are arranged in columns and bands.

The qEEG oscillatory band results showed that Compound 1 dosing led to an immediate limited decrease of delta amplitude followed by a longer-lasting rebound increase which appeared to accumulate with repeated dosing, resulting in a prominent increase in delta amplitude with short transient normalizations after each additional dose. The delta increase appeared to be more prominent over the right and posterior sides. The carry-over effects on Day 16 suggest that the accumulation is strongest in Cohort 4A for EO, and Cohort 2 for EC.

After repeated dosing, a sustained global decrease of EO and EC theta band amplitudes was consistently observed pre-dose. Alpha 1 band amplitude decreased after single and repeated dosing of Compound 1 in Cohorts 1 and 3, but a tendency developed towards a posterior increase in alpha 1 band amplitude for EO in Cohorts 2 and 4A and for EC in Cohort 4B. The pattern of change for alpha 2 band amplitude aligned with the effects for alpha 1 band amplitudes, with a progressive decrease of alpha 2 over days.

Cohorts 2 and 4B showed a prominent increase in EO beta 2 band amplitude building up over the 10 treatment days. A global decrease in EC beta 3 band amplitude was observed, except for the temporal region in Cohort 4B. Findings for EO beta 3 band amplitudes were similar, but less consistent.

Cohort 2 showed decreased EC ASI (alpha slow wave index) over time. Cohorts 2 and 4A showed a decrease of EO ASI with multiple dosing, while an increase was observed for Cohort 4B. An increase in EC TBR (theta/beta ratio) was observed over the posterior region for Cohort 2, while a decrease was observed at later timepoints. EC BAR (beta/alpha ratio) tended to increase transiently after dosing in Cohorts 2 and 4A, with a sustained increase observed on Day 16; a rostral increase in EO BAR was observed on Day 3 and Day 10.

The outstanding feature of the fractal changes observed by qEEG was an early, post-dose (D1-5, +1-4 h) increase in EC gamma band amplitudes, which diminished or was even replaced by a late (+8 h) decrease, which also manifested as a rostral pre-dose decrease on Day 10 for all Cohorts. qEEG results on Day 16 still showed a rostral decrease in gamma band amplitude (most evident for Cohort 2), often combined with a caudal increase. TBR and 1/f were observed to change in parallel with gamma band amplitudes.

The results demonstrated early effects of Compound 1 on EEG activity, as well as sustained, dose-dependent effects on EEG activity after continuous dosing, indicating successful receptor engagement in the brain.

### Genotyping

A blood sample was collected to allow for investigation into genetic polymorphisms for drug-metabolizing enzymes and drug transporter proteins that could impact the PKs of Compound 1 and its metabolites.

### Bladder Ultrasounds

An ultrasound of the bladder was taken within 10 minutes of the bladder being voided. Postvoid residual (PVR) urine volume was recorded.

### Safety and Tolerability

Favorable safety and tolerability results were observed in all cohorts.

Although the disclosure has been described with reference to the above examples, it will be understood that modifications and variations are encompassed within the spirit and scope of the disclosure. The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1-51. (canceled)

52. A method of treating or preventing a 5-hydroxytryptamine (HT)_{2C} receptor-associated disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered twice daily.

53. The method of claim 52, wherein the 5-hydroxytryptamine (HT)_{2C} receptor-associated disorder is epilepsy.

54. The method of claim 52, wherein the administering results in a reduction in severity of an epileptic seizure in the patient.

55. The method of claim 52, wherein the administering results in a reduction in the frequency of epileptic seizures in the patient.

56. The method of claim 53, wherein the epilepsy is a refractory epilepsy.

57. A method of treating or preventing a seizure disorder in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered twice daily.

58. The method of claim 57, wherein the seizure disorder is selected from epilepsy, epilepsy with generalized tonic-clonic seizures, epilepsy with myoclonic absences, frontal lobe epilepsy, temporal lobe epilepsy, Landau-Kleffher Syndrome, Rasmussen's syndrome, Dravet syndrome, Doose syndrome (epilepsy with myoclonic atonic seizures (EM AS)), CDKL5 deficiency disorder (CDKL5 encephalopathy, or CDD), infantile spasms (West syndrome), juvenile myoclonic epilepsy (JME), vaccine-related encephalopathy, intractable childhood epilepsy (ICE), Lennox-Gastaut syndrome (LGS), Rett syndrome, Ohtahara syndrome (early infantile DEE, EIDEE), childhood absence epilepsy, essential tremor, acute repetitive seizures, benign rolandic epilepsy, status epilepticus, refractory status epilepticus, super-refractory status epilepticus (SRSE), PCDH19 pediatric epilepsy, drug withdrawal induced seizures, alcohol withdrawal induced seizures, increased seizure activity and breakthrough seizures.

59. A method of treating or preventing developmental and epileptic encephalopathy (DEE) in a patient in need thereof, wherein the method comprises administering to the patient (R)—N-(2,2-difluoroethyl)-7-methyl-1,2,3,4,6,7-hexahydro-[1,4]diazepino[6,7,1-hi]indole-8-carboxamide (Compound 1), or a pharmaceutically acceptable salt thereof,
wherein Compound 1, or a pharmaceutically acceptable salt thereof, is administered twice daily.

60. The method of claim 59, wherein the DEE is selected from Lennox-Gastaut syndrome, Dravet syndrome, Doose syndrome (EM AS), West syndrome (infantile spasms), Landau-Kleffner syndrome, and genetic disorders such as CDKL5 encephalopathy (CDK5L deficiency disorder) or CHD2 encephalopathy.

61. The method of claim 60, wherein the DEE is selected from Ohtahara syndrome (EIDEE), Lennox-Gastaut syndrome, Dravet syndrome, Doose syndrome (EM AS), West syndrome (infantile spasms), Landau-Kleffner syndrome, tuberous sclerosis complex, CDKL5 encephalopathy (CDKL5 deficiency disorder), dup15q syndrome, SCN2A related epilepsies, SCN8A related epilepsies, KCNQ2 related epilepsies, KCNQ3 related epilepsies, Angelman syndrome, KCNT1 related epilepsies, SynGAP1 related epilepsies, Rett syndrome, PCDH19 epilepsy, ring 14 syndrome, ring 20 syndrome, CHD2 encephalopathy, early myoclonic encephalopathy, epilepsy of infancy with migrating focal seizures, and epileptic encephalopathy with continuous spike-wave.

62. The method of claim 52, wherein Compound 1, or a pharmaceutically acceptable salt thereof, is administered in a dosage equivalent to about 3 mg/dose of Compound 1, or about 6 mg/dose of Compound 1, or about 12 mg/dose of Compound 1, or about 15 mg/dose of Compound 1, or about 18 mg/dose of Compound 1.

63. The method of claim 52, wherein Compound 1, or a pharmaceutically acceptable salt thereof, is administered in a dosage equivalent to about 12 mg/dose.

64. The method of claim 52, wherein Compound 1, or a pharmaceutically acceptable salt thereof, is administered in a dosage equivalent to about 15 mg/dose.

65. The method of claim 52, wherein Compound 1, or a pharmaceutically acceptable salt thereof, is administered in a dosage equivalent to about 18 mg/dose.

66. The method of claim 52, wherein Compound 1, or a pharmaceutically acceptable salt thereof, is administered via a titration scheme that comprises the up-titration of Compound 1, or a pharmaceutically acceptable salt thereof, until an optimized dosage is administered.

67. The method of claim 52, wherein the administration results in an improvement in the frequency of convulsive/motor seizures.

68. The method of claim 52, the administration results in an improvement in one or more of:
frequency of observed countable motor seizures;
number of total seizures;
frequency of non-convulsive seizure;
number of episodes of status epilepticus;
frequency of use of rescue medication; and
number of countable motor seizure-free days.

69. The method of claim 52, wherein the method provides improvement in at least one symptom selected from ataxia, gait impairment, speech impairment, vocalization, impaired cognition, abnormal motor activity, clinical seizure, subclinical seizure, hypotonia, hypertonia, drooling, mouthing behavior, aura, convulsions, repetitive movements, unusual sensations, frequency of seizures and severity of seizures.

70. The method of claim 52, wherein the Compound 1, or a pharmaceutically acceptable salt thereof, is an HCl salt of Compound 1.

71. The method of claim 52, wherein the administration results in a ratio of a geometric mean steady-state C_{trough} of Compound 1 in CSF to a geometric mean steady-state C_{trough} of Compound 1 in plasma (CSF/P C_{trough}) of at least about 1.4.
